(19) United States
(12) Patent Application Publication
(10) Pub. No.: US 2009/0082332 A1
(43) Pub. Date: Mar. 26, 2009

(54) PURINE DERIVATIVES FOR THE TREATMENT OF VIRAL OR ALLERGIC DISEASES AND CANCERS

(76) Inventors: Philip Abbot, Leicestershire (GB); Roger Victor Bonnert, Leicestershire (GB); Stephen Brough, Leicestershire (GB); Kamaldeep Chohan, Leicestershire (GB); Thomas McInally, Leicestershire (GB); Stephen Thom, Leicestershire (GB); Yoshiaki Isobe, Osaka (JP); Kei Nakamura, Osaka (JP); Shingo Tojo, Osaka (JP)

Correspondence Address: MORGAN LEWIS & BOCKIUS LLP, 1111 PENNSYLVANIA AVENUE NW, WASHINGTON, DC, 20004, US

(21) Appl. No.: 12/067,536

(22) PCT Filed: Sep. 20, 2006
(86) PCT No.: PCT/GB2006/003490

§ 371 Date: Sep. 15, 2008

(30) Foreign Application Priority Data
Sep. 22, 2005 (SE) 0502101-9

(51) Int. Cl. A61K 31/397; C07D 473/02; A61P 31/12; A61P 35/00; A61P 37/08; A61K 31/522

(52) U.S. Cl. 514/210.21; 544/276; 514/263.22; 514/263.2

(57) ABSTRACT

The present invention provides compounds of formula (I)

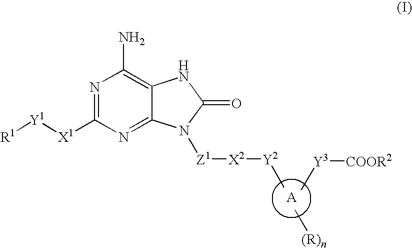

wherein R¹, Y¹, X¹, Z¹, X², Y², A, Y³, n, R and R² are as defined in the specification, processes for their preparation, pharmaceutical compositions containing them and their use in therapy.

The present invention relates to adenine derivatives, processes for their preparation, pharmaceutical compositions containing them and their use in therapy.

The immune system is comprised of innate and acquired immunity, both of which work cooperatively to protect the host from microbial infections. It has been shown that innate immunity can recognize conserved pathogen-associated molecular patterns through toll-like receptors (TLRs) expressed on the cell surface of immune cells. Recognition of invading pathogens then triggers cytokine production (including interferon alpha(IFNα)) and upregulation of co-stimulatory molecules on phagocytes, leading to modulation of T cell function. Thus, innate immunity is closely linked to acquired immunity and can influence the development and regulation of an acquired response.

TLRs are a family of type I transmembrane receptors characterized by an NH₂-terminal extracellular leucine-rich repeat domain (LRR) and a COOH-terminal intracellular tail containing a conserved region called the Toll/IL-1 receptor (TIR) homology domain. The extracellular domain contains a varying number of LRR, which are thought to be involved in ligand binding. Eleven TLRs have been described to date in humans and mice. They differ from each other in ligand specificities, expression patterns, and in the target genes they can induce.

Ligands which act via TLRs (also known as immune response modifiers (IRMS)) have been developed, for example, the imidazoquinoline derivatives described in U.S. Pat. No. 4,689,338 which include the product Imiquimod for treating genital warts, and the adenine derivatives described in WO 98/01448 and WO 99/28321.

This patent application describes a class of 9-substituted-8-oxoadenine compounds having immuno-modulating properties which act via TLR7 that are useful in the treatment of viral or allergic diseases and cancers.

In accordance with the present invention, there is therefore provided a compound of formula (I):

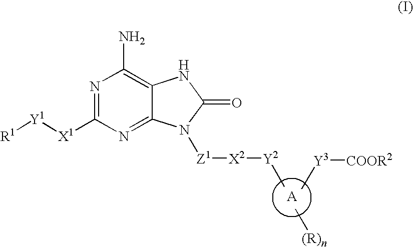

wherein

R¹ represents hydrogen, hydroxyl, C₁-C₆ alkoxy, C₂-C₅ alkoxycarbonyl, C₁-C₆ haloalkyl, C₁-C₆ haloalkoxy, or a C₆-C₁₀ aryl, C₅-C₁₀ heteroaryl or C₃-C₈ cycloalkyl group, each group being optionally substituted by one or more substituents independently selected from halogen, hydroxyl, C₁-C₆ alkyl, C₁-C₆ haloalkyl, C₁-C₆ alkoxy, C₁-C₆ haloalkoxy, C₂-C₅ alkoxycarbonyl, amino (NH₂) and (di)-C₁-C₆ alkylamino;

Y¹ represents a single bond or C₁-C₆ alkylene;

X¹ represents a single bond or an oxygen or sulphur atom or sulphonyl (SO₂) or NR³;

Z¹ represents a C₂-C₆ alkylene or C₃-C₈ cycloalkylene group, each of which may be optionally substituted by at least one hydroxyl;

X² represents NR⁴, CONR⁴, NR⁴CO, SO₂NR⁴, NR⁴SO₂, NR⁴CONR⁵ or NR⁵CONR⁴;

Y² represents a single bond or C₁-C₆ alkylene;

Y³ represents a single bond or C₁-C₆ alkylene;

n is an integer 0, 1 or 2;

each R independently represents halogen, C₁-C₆ alkyl, C₁-C₆ hydroxyalkyl, C₁-C₆ haloalkyl, C₁-C₆ alkoxy, C₁-C₆ hydroxyalkoxy, C₁-C₆ haloalkoxy, amino (NH₂), (di)-C₁-C₆ alkylamino, C₁-C₆ alkylamino or a C₄-C₇ saturated heterocyclic ring comprising a ring nitrogen atom and optionally one or more further heteroatoms independently selected from nitrogen, oxygen and sulphur, the heterocyclic ring being optionally substituted by one or more substituents independently selected from halogen, hydroxyl, oxo, C₁-C₆ alkyl, C₁-C₆ alkoxy, C₂-C₅ alkylcarbonyl and C₂-C₅ alkoxycarbonyl;

R² represents hydrogen or a C₁-C₆ alkyl, C₂-C₆ alkenyl, C₂-C₆ alkynyl or C₃-C₈ cycloalkyl group, each group being optionally substituted by one or more substituents independently selected from halogen, hydroxyl, C₁-C₆ alkoxy, C₂-C₁₀ acyloxy, amino (NH₂), (di)-C₁-C₆ alkylamino and a C₄-C₇ saturated heterocyclic ring comprising a ring nitrogen atom and optionally one or more further heteroatoms independently selected from nitrogen, oxygen and sulphur, the heterocyclic ring in turn being optionally substituted by one or more substituents independently selected from halogen, hydroxyl, oxo, C₁-C₆ alkyl, C₁-C₆ alkoxy, C₂-C₅ alkylcarbonyl and C₂-C₅ alkoxycarbonyl;

R³ represents hydrogen or C₁-C₆ alkyl;

R⁴ represents a 3- to 8-membered saturated heterocyclic ring comprising a ring group NR⁶;

R⁵ represents hydrogen or a C₁-C₆ alkyl or C₃-C₆ cycloalkyl group, each of which may be optionally substituted by one or more substituents independently selected from halogen, hydroxyl and NR⁷R⁸;

R⁶ represents hydrogen, CO₂R⁹, SO₂R⁹, COR⁹, SO₂NR¹⁰R¹¹, CONR¹⁰R¹¹, a 3- to 8-membered saturated heterocyclic ring comprising a ring group NR⁹, or

(i) a C₆-C₁₀ aryl or C₅-C₁₀ heteroaryl group, each of which may be optionally substituted by one or more substituents independently selected from halogen, cyano, oxo, carboxyl, S(O)ₘR¹², OR¹³, SO₂NR¹³R¹⁴CONR¹³R¹⁴, NR¹³R¹⁴, NR¹³SO₂R¹², NR¹³CO₂R¹², NR¹³COR¹², C₁-C₆ alkyl and C₁-C₃ haloalkyl, or
(ii) a C₁-C₆ alkyl, C₂-C₆ alkenyl, C₂-C₆ alkynyl or C₃-C₈ cycloalkyl group, each of which may be optionally substituted by one or more substituents independently selected from halogen, cyano, C₃-C₈ cycloalkyl, OR¹⁵, S(O)ₚR¹⁶, CO₂R¹⁷, NR¹⁸R¹⁹, CONR¹⁸R¹⁹, NR¹⁸COR¹⁶, SO₂NR¹⁸R¹⁹, NR¹⁸SO₂R¹⁶ and a group as defined in (i) above;

R⁷ and R⁸ each independently represent hydrogen, C₁-C₆ alkyl or C₃-C₆ cycloalkyl, or

R⁷ and R⁸ together with the nitrogen atom to which they are attached form a 3- to 8-membered saturated heterocyclic ring comprising at least one heteroatom or heterogroup selected from nitrogen, oxygen, sulphur and sulphonyl, the heterocyclic ring being optionally substituted by one or more substituents independently selected from halogen, hydroxyl, carboxyl, cyano, OR²³, S(O)_{q}R²³, NR²⁴R²⁵, C₁-C₆ alkyl and C₃-C₈ cycloalkyl;

R¹³, R¹⁴, R¹⁵, R¹⁷, R²⁰, R²¹, R²⁴, R²⁵, R²⁶ and R²⁷ each independently represent hydrogen, C₁-C₆ alkyl or C₃-C₆ cycloalkyl;

R⁹, R¹⁶ and R²³ each independently represent a C₁-C₆ alkyl or C₃-C₆ cycloalkyl group, each of which may be optionally substituted by one or more substituents independently selected from halogen, carboxyl, hydroxyl and NR²⁰R²¹;

either R¹⁰ represents hydrogen or a C₁-C₆ alkyl, C₂-C₆ alkenyl, C₂-C₆ alkynyl or C₃-C₈ cycloalkyl group, each of which may be optionally substituted by one or more substituents independently selected from halogen, hydroxyl, carboxyl, cyano, OR²³, S(O)_{q}R²³, NR²⁴R²⁵ and C₃-C₈ cycloalkyl, and

R¹¹ represents hydrogen or a C₁-C₆ alkyl or C₃-C₆ cycloalkyl group, each of which may be optionally substituted by one or more substituents independently selected from halogen, hydroxyl and NR²⁶R²⁷, or

R¹⁰ and R¹¹ together with the nitrogen atom to which they are attached form a 3 to 8-membered saturated heterocyclic ring comprising at least one heteroatom or heterogroup selected from nitrogen, oxygen, sulphur and sulphonyl, the heterocyclic ring being optionally substituted by one or more substituents independently selected from halogen, hydroxyl, carboxyl, cyano, OR²³, S(O)_{q}R²³, NR²⁴R²⁵, C₁-C₆ alkyl and C₃-C₈ cycloalkyl;

R¹² represents C₁-C₆ alkyl or C₃-C₆ cycloalkyl;

R¹⁸ and R¹⁹ are defined as for R¹⁰ and R¹¹ respectively;

m, p and q each independently represent an integer 0, 1 or 2; and

A represents a C₆-C₁₀ aryl or a C₅-C₁₂ heteroaryl group;

or a pharmaceutically acceptable salt or solvate thereof.

In the context of the present specification, unless otherwise stated, an alkyl substituent group or an alkyl moiety in a substituent group may be linear or branched. Examples of C₁-C₆ alkyl groups/moieties include methyl, ethyl, n-propyl, isopropyl, n-butyl, isobutyl, tert-butyl, n-pentyl and n-hexyl. Similarly, an alkylene group/moiety may be linear or branched. Examples of C₁-C₆ alkylene groups/moieties include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. A C₁-C₆ haloalkyl or C₁-C₆ haloalkoxy substituent group/moiety will comprise at least one halogen atom, e.g. one, two, three, four or five halogen atoms, examples of which include trifluoromethyl, trifluoromethoxy or pentafluoroethyl. The alkyl groups in a di-C₁-C₆ alkylamino or alkylcarbonyl group/moiety may be the same as, or different from, one another. A C₁-C₆ hydroxyalkyl or C₁-C₆ hydroxyalkoxy substituent group/moiety will comprise at least one hydroxyl group, e.g. one, two or three hydroxyl groups. An aryl or heteroaryl substituent group/moiety may be monocyclic or polycyclic (e.g. bicyclic or tricyclic) in which the two or more rings are fused. A heteroaryl group/moiety will comprise at least one ring heteroatom (e.g. one, two, three or four ring heteroatoms independently) selected from nitrogen, oxygen and sulphur. Examples of aryl and heteroaryl groups/moieties include phenyl, 1-naphthyl, 2-naphthyl, furyl, thienyl, pyrrolyl, pyridyl, indolyl, isoindolyl, quinolyl, isoquinolyl, pyrazolyl, imidazolyl, pyrimidinyl, pyrazinyl, pyridazinyl, thiazolyl and oxazolyl.

A C₂-C₁₀ acyloxy group/moiety is exemplified by a C₂-C₅ alkylcarbonyloxy group, a C₂-C₅ alkenylcarbonyloxy group, a C₂-C₅ alkynylcarbonyloxy group, a C₆-C₉ arylcarbonyloxy group or a C₅-C₉ heteroarylcarbonyloxy group, each of which may be optionally substituted by one or more substituents independently selected from halogen, hydroxyl, C₁-C₃ alkoxy or phenyl ring, optionally substituted by from halogen, hydroxyl, cyano, OR²³, S(O)_{q}R²³ or C₁-C₆ alkyl, providing that the total number of carbon atoms in the acyloxy group does not exceed 10.

Preferably R¹ represents hydrogen.

Preferably Y¹ represents C₁-C₆ alkylene, more preferably C4 alkylene

Preferably X¹ represents oxygen

Preferably Z¹ represents C₂-C₆ alkylene, more preferably (CH₂)₃.

Preferably X² represents NR⁴. Preferably R⁴ is a 4 to 6-membered saturated heterocyclic ring comprising a ring group NR⁶. Preferred R⁶ groups include those exemplified herein, such as hydrogen, COMe, (CH₂)₂OH, (CH₂)₃OH, methyl, ethyl, CH₂CO₂-t-butyl, CH₂CO₂H, benzyl, CH₂CO₂Me, iso-propyl, iso-butyl, CH₂CN, (CH₂)₂CN, (CH₂)₃CN, (CH₂)₃CO₂butyl, and (CH₂)₃CO₂H.

Preferably Y² represents C₁-C₆ alkylene, more preferably a CH₂ group.

Preferably A represents a C₆-C₁₀ aryl, more preferably phenyl.

Preferably R is hydrogen.

Preferably Y³ represents C₁-C₆ alkylene, more preferably CH₂.

Preferably R² represents C₁-C₆ alkyl more preferably methyl.

Examples of compounds of the invention include
Methyl (3-{[[3-(6-amino-2-butoxy-8-oxo-7,8-dihydro-9H-purin-9-yl)propyl](piperidin-4-yl)amino]methyl}phenyl)acetate,
Methyl [3-({(1-acetylpiperidin-4-yl)[3-(6-amino-2-butoxy-8-oxo-7,8-dihydro-9H-purin-9-yl)propyl]amino}methyl)phenyl]acetate,
Methyl [3-({[3-(6-amino-2-butoxy-8-oxo-7,8-dihydro-9H-purin-9-yl)propyl][1-(2-hydroxyethyl)piperidin-4-yl]amino}methyl)phenyl]acetate,
Methyl [3-({[3-(6-amino-2-butoxy-8-oxo-7,8-dihydro-9H-purin-9-yl)propyl][1-(3-hydroxypropyl)piperidin-4-yl]amino}methyl)phenyl]acetate,
Methyl (3-{[[3-(6-amino-2-butoxy-8-oxo-7,8-dihydro-9H-purin-9-yl)propyl](1-methylpiperidin-4-yl)amino]methyl}phenyl)acetate,
Methyl (3-{[[3-(6-amino-2-butoxy-8-oxo-7,8-dihydro-9H-purin-9-yl)propyl](1-ethylpiperidin-4-yl)amino]methyl}phenyl)acetate,
Methyl [3-({[3-(6-amino-2-butoxy-8-oxo-7,8-dihydro-9H-purin-9-yl)propyl][1-(2-tert-butoxy-2-oxoethyl)piperidin-4-yl]amino}methyl)phenyl]acetate,
(4-{[3-(6-Amino-2-butoxy-8-oxo-7,8-dihydro-9H-purin-9-yl)propyl][3-(2-methoxy-2-oxoethyl)benzyl]amino}piperidin-1-yl)acetic acid,
Methyl (3-{[[3-(6-amino-2-butoxy-8-oxo-7,8-dihydro-9H-purin-9-yl)propyl](1-benzylpiperidin-4-yl)amino]methyl}phenyl)acetate,
Methyl (3-{[[4-(6-amino-2-butoxy-8-oxo-7,8-dihydro-9H-purin-9-yl)butyl](1-methylpiperidin-4-yl)amino]methyl}phenyl)acetate,
Methyl (3-{2-[[3-(6-amino-2-butoxy-8-oxo-7,8-dihydro-9H-purin-9-yl)propyl](1-methylpiperidin-4-yl)amino]-2-oxoethyl}phenyl)acetate,
Methyl [3-({[3-(6-amino-2-butoxy-8-oxo-7,8-dihydro-9H-purin-9-yl)propyl][(3R)-1-benzylpyrrolidin-3-yl]amino}methyl)phenyl]acetate,
Methyl (3-{[[3-(6-amino-2-butoxy-8-oxo-7,8-dihydro-9H-purin-9-yl)propyl](1-isopropylpiperidin-4-yl)amino]methyl}phenyl)acetate,
Methyl [3-({[3-(6-amino-2-butoxy-8-oxo-7,8-dihydro-9H-purin-9-yl)propyl][1-(cyanomethyl)piperidin-4-yl]amino}methyl)phenyl]acetate,
Methyl [3-({[3-(6-amino-2-butoxy-8-oxo-7,8-dihydro-9H-purin-9-yl)propyl][1-(2-cyanoethyl)piperidin-4-yl]amino}methyl)phenyl]acetate,
Methyl [3-({[3-(6-amino-2-butoxy-8-oxo-7,8-dihydro-9H-purin-9-yl)propyl][1-(3-cyanopropyl)piperidin-4-yl]amino}methyl)phenyl]acetate, tert-Butyl 4-(4-{[3-(6-amino-2-butoxy-8-oxo-7,8-dihydro-9H-purin-9-yl)propyl][3-(2-methoxy-2-oxoethyl)benzyl]amino}piperidin-1-yl)butanoate,
4-(4-{[3-(6-Amino-2-butoxy-8-oxo-7,8-dihydro-9H-purin-9-yl)propyl][3-(2-methoxy-2-oxoethyl)benzyl]amino}piperidin-1-yl)butanoic acid,
Methyl (3-{[{3-[6-amino-2-(2-methoxyethoxy)-8-oxo-7,8-dihydro-9H-purin-9-yl]propyl}(1-methylpiperidin-4-yl)amino]methyl}phenyl)acetate,
Methyl (4-{[[3-(6-amino-2-butoxy-8-oxo-7,8-dihydro-9H-purin-9-yl)propyl](1-methylazetidin-3-yl)amino]methyl}phenyl)acetate,
Methyl (4-{[[3-(6-amino-2-butoxy-8-oxo-7,8-dihydro-9H-purin-9-yl)propyl](1-ethylazetidin-3-yl)amino]methyl}phenyl)acetate,
Methyl (4-{[[3-(6-amino-2-butoxy-8-oxo-7,8-dihydro-9H-purin-9-yl)propyl](1-isopropylazetidin-3-yl)amino]methyl}phenyl)acetate,
Methyl (4-{[[3-(6-amino-2-butoxy-8-oxo-7,8-dihydro-9H-purin-9-yl)propyl](1-isobutylazetidin-3-yl)amino]methyl}phenyl)acetate,
Methyl [4-({[3-(6-amino-2-butoxy-8-oxo-7,8-dihydro-9H-purin-9-yl)propyl][(3R)-1-methylpyrrolidin-3-yl]amino}methyl)phenyl]acetate,
Methyl (4-{[[3-(6-amino-2-butoxy-8-oxo-7,8-dihydro-9H-purin-9-yl)propyl](1′-methyl-1,4′-bipiperidin-4-yl)amino]methyl}phenyl)acetate,
Methyl (4-{[[3-(6-amino-2-butoxy-8-oxo-7,8-dihydro-9H-purin-9-yl)propyl](1-propylazetidin-3-yl)amino]methyl}phenyl)acetate
Methyl [4-({[3-(6-amino-2-butoxy-8-oxo-7,8-dihydro-9H-purin-9-yl)propyl][(38)-1-methylpyrrolidin-3-yl]amino}methyl)phenyl]acetate
   and pharmaceutically acceptable salts of any one thereof.

The present invention further provides a process for the preparation of a compound of formula (I).

Compounds of formula (I) where X² represents NR⁴ may be prepared by reacting a compound of formula (II)

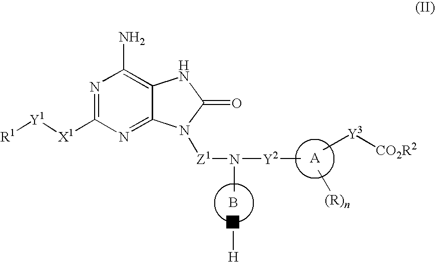

wherein n, Y¹, Y², Y³, X¹, A, Z¹, R, R¹ and R² are as defined in formula (I) and B is defined as a 3- to 8-membered saturated heterocyclic ring comprising a ring group NH, with a compound of formula

L¹-R⁶ (III)

wherein L¹ represents a leaving group (e.g. halogen, mesylate or triflate) and R⁶ is as defined in formula (I),
and optionally after carrying out one or more of the following:
converting the compound obtained to a further compound of the invention
removal of any protecting groups
forming a pharmaceutically acceptable salt of the compound.

The reaction may conveniently be carried out in an organic solvent such as NMP, DMF, acetonitrile or tetrahydrofuran usually in the presence of a suitable base (e.g. triethylamine, sodium carbonate or potassium carbonate) at a temperature, for example, in the range from 0 to 150° C.

Alternatively, a compound of formula (I) where X² represents NR⁴ may be prepared by reacting a compound of formula (II) with an appropriate aldehyde or ketone in the presence of a reducing agent such as sodium triacetoxyborohydride or sodium cyano borohydride.

(a) A compound of formula (II) may be prepared by reacting a compound of formula (IV)

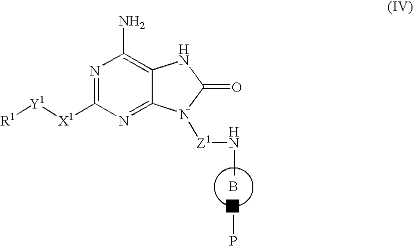

wherein Y¹, X¹, Z¹, R¹ and B are as defined in formula (II) and P is a nitrogen protecting group (e.g. tert-butoxycarbonyl), with a compound of formula (V)

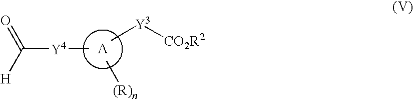

wherein Y⁴ represents a bond or a C₁-C₅ alkylene group and n, A, Y³, R and R² are as defined in formula (I) in the presence of a suitable reducing agent (e.g. sodium triacetoxyborohydride); or
(b) reacting a compound of formula (IV) as defined in (a) above with a compound of formula

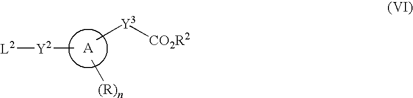

wherein L² represents a leaving group (e.g. halogen, mesylate or triflate) and n, A, Y², Y³, R and R² are as defined in formula (I) in the presence of a suitable base (e.g. sodium carbonate or potassium carbonate)

In process (a), the reaction may conveniently be carried out in an organic solvent such as 1-methyl-2-pyrrolidinone, 1,2-dichloroethane or tetrahydrofuran at a temperature, for example, in the range from 0 to 150° C.

In process (b), the reaction may conveniently be carried out in an organic solvent such as acetonitrile, 1-methyl-2-pyrrolidinone or N,N-dimethylformamide at a temperature, for example, in the range from 0 to 150° C.

Following process (a) or (b) the nitrogen protecting group is removed using known literature methods

A compound of formula (IV) may be prepared by reacting a compound of formula (VII)

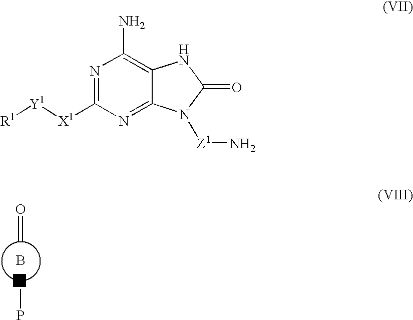

wherein Y¹, X¹, Z¹ and R¹ are as defined in formula (I), with a compound of formula (VIII), where B is defined as a 3- to 8-membered saturated heterocyclic ring and P is defined as a nitrogen protecting group (e.g. tert-butoxycarbonyl).

The reaction may conveniently be carried out in an organic solvent such as NMP, 1,2-dichloroethane, methanol or tetrahydrofuran at a temperature, for example, in the range from 0 to 150° C. in the presence of a reducing agent (e.g. sodium triacetoxyborohydride or sodium cyanoborohydride). The presence of an acid, such as acetic acid, may also be advantageous.

Alternatively, a compound of formula (II) may be prepared by reacting a compound of formula (IX)

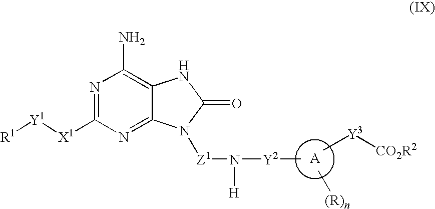

wherein n, Y¹, Y², Y³, X¹, A, Z¹, R, R¹ and R² are as defined in formula (I), with a compound of formula (VIII), followed by deprotection of the nitrogen protecting group, under the same conditions described for the preparation of a compound of formula (IV).

A compound of formula (IX) may be prepared by reacting a compound of formula (VII) with a compound of formula (V) or (VI) under the same condition as described in (a) and (b).

A compound of formula (I) may be prepared by reacting a compound of formula (IX) with a compound of formula (X)

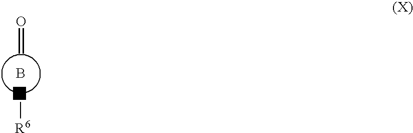

where B is defined as a 3- to 8-membered saturated heterocyclic ring and R⁶ is defined as in formula (I), under the same conditions described for the preparation of a compound of formula (IV) in process (a).

A compound of formula (I) may be prepared by reacting a compound of formula (XI)

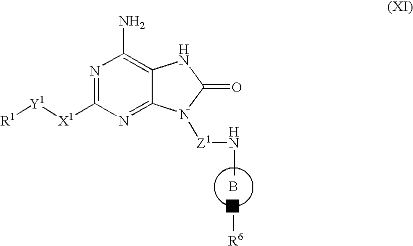

wherein Y¹, X¹, Z¹, R¹ and R⁶ are as defined in formula (I), B is defined as a 3- to 8-membered saturated heterocyclic ring, with a compound of formula (V) or (VI) under the same conditions described for the preparation of a compound of formula (IV).

Compounds of formula (IV) or (XI), where X¹ represents O may be prepared as illustrated in the following reaction scheme:

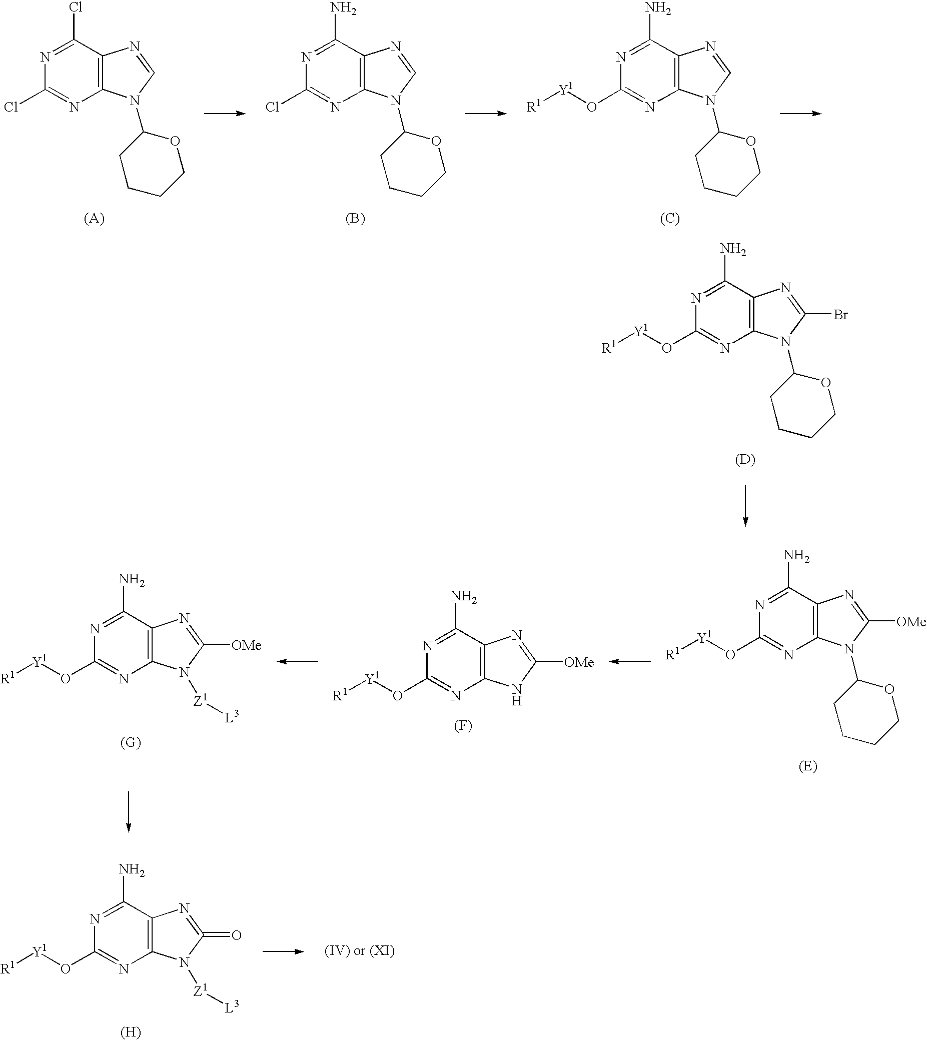

The compound of formula (B) is prepared by reacting the compound of formula (A) with ammonia in an organic solvent such as methanol, ethanol, propanol, butanol, tetrahydrofuran, 1,4-dioxane, diglyme, acetonitrile or an aqueous mixture of any one of the preceding solvents. The reaction may be carried out in an autoclave, and at a temperature, for example, in the range from 20 to 200° C.

Compounds of formula (C) may be prepared by reacting the compound of formula (B) with an alcohol of formula

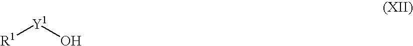

in the presence of a base such as sodium hydride and in an organic solvent such as tetrahydrofuran, 1,4-dioxane, diglyme, N,N-dimethylformamide or dimethylsulfoxide, preferably at elevated temperature, e.g. at a temperature in the range from 20 to 150° C. Alternatively an alkali metal such as sodium may be dissolved in a C₁-C₆ alkanol and then reacted with the compound of formula (B), preferably at elevated temperature, e.g. at a temperature in the range from 20 to 150° C.

Compounds of formula (D) are prepared by brominating a compound of formula (C). The reaction may be carried out using a brominating agent such as bromine, hydroperbromic acid or N-bromosuccinimide, in an organic solvent such as carbon tetrachloride, methylene chloride, dichloroethane, diethyl ether, acetic acid or carbon disulfide. The reaction temperature will generally be in the range from 0° C. to the boiling point of the solvent.

Compounds of formula (E) are prepared by reacting a compound of formula (D) with sodium methoxide in an organic solvent such as methanol and at a temperature, for example, in the range from 20 to 150° C.

Compounds of formula (F) may be obtained by treating a compound of formula (E) with an acid such as trifluoroacetic acid in an organic solvent such as methanol. Compounds of formula (G) are prepared by reacting a compound of formula (F) with a compound of formula L³-Z¹-L³ wherein L³ represents a leaving group such as a halogen, mesylate or triflate and Z¹ is as defined in formula (I). The reaction may be carried out in an organic solvent such as N,N-dimethylformamide, dimethylsulfoxide or acetonitrile with a base present, preferably at room temperature (20° C.). A base such as an alkali metal carbonate, e.g. sodium carbonate or potassium carbonate; an alkaline earth metal carbonate, e.g. calcium carbonate; a metal hydroxide, e.g. sodium hydroxide or potassium hydroxide; a metal hydrogenate, e.g. sodium hydride; or a metal alkoxide, e.g. potassium t-butoxide, may be used.

Compounds of formula (H) may be obtained by treatment of a compound of formula (G) with an acid. The reaction may be carried out in an organic solvent such as methanol using either an inorganic acid such as hydrochloric acid, hydrobromic acid or sulfuric acid, or an organic acid such as trifluoroacetic acid.

Compounds of formula (IV) or (XI) may be prepared by reacting a compound of formula (H) with an amine of formula (XIII) or (XIV).

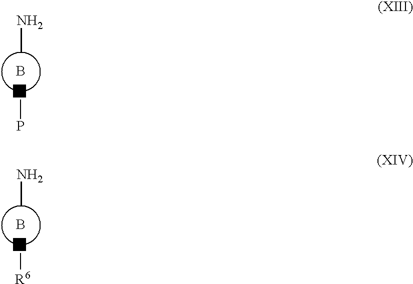

wherein R⁶ is as defined in formula (I), B is defined as a 3- to 8-membered saturated heterocyclic ring and P is a nitrogen protecting group.

The reaction may be carried out in an organic solvent such as acetonitrile or N,N-dimethylformamide using an excess of the amine, preferably at elevated temperature, e.g. at a temperature in the range from 0 to 150° C.

Compounds of formula (IV) and (XI) may also be prepared by reacting a compound of formula (VIII) or (X) with a compound of formula

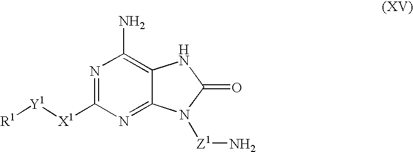

wherein Y¹, X¹, Z¹ and R¹ are as defined in formula (I), under the same conditions described for the preparation of a compound of formula (IV) in process (a).

Compounds of formula (XV) may be obtained by reacting a compound of formula (F) as defined above with a compound of formula (XVI), L⁴-Z-N¹—P, wherein L⁴ represents a leaving group (e.g. halogen, mesylate or triflate), P represents a nitrogen-protecting group (e.g. butoxycarbonyl) and Z¹ is as defined in formula (I), followed by removal of the nitrogen-protecting group, P, and removal of the oxygen-protecting group in the substituent —OCH₃.

The reaction between the compounds of formula (F) and (XVI) may be carried out in an organic solvent such as N,N-dimethylformamide, dimethylsulfoxide or acetonitrile with a base present, at a temperature, for example, in the range from 0 to 150° C. The base used may be an alkali metal carbonate, e.g. sodium carbonate or potassium carbonate; an alkaline earth metal carbonate, e.g. calcium carbonate; a metal hydroxide, e.g. sodium hydroxide or potassium hydroxide; a metal hydrogenate, e.g. sodium hydride; or a metal alkoxide, e.g. potassium tert-butoxide. The removal of the protecting groups may be carried out according to methods known in the art.

Compounds of formula (I) where X² represents NR⁴CO or NR⁴SO₂ may be prepared by reacting a compound of formula (XI) with a compound of formula (XVII) or (XVIII)

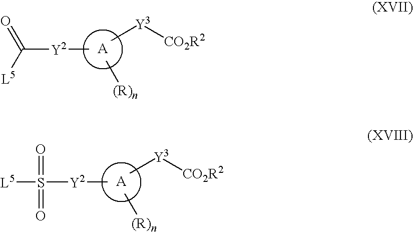

wherein n, A, Y², Y³, R and R² are as defined in formula (I), L⁵ represents a leaving group such as a halogen, or an activated hydroxyl (for example treating a carboxylic acid with a coupling reagent such as EDC or HATU)

When L⁵ represents halogen the reaction may be carried out in an organic solvent such as DCM with a base such as triethylamine or pyridine, preferably at a temperature in the range from 0 to the boiling point of the solvent.

When L⁵ represents an activated hydroxyl, the reaction may be carried out in an organic solvent such as DMF or THF, preferably at a temperature in the range from 0 to 50° C. Additives such as HOBt and a base such as N,N-diisopropylethylamine may be advantageous.

A compound of formula (I) may also be prepared by the route shown below;

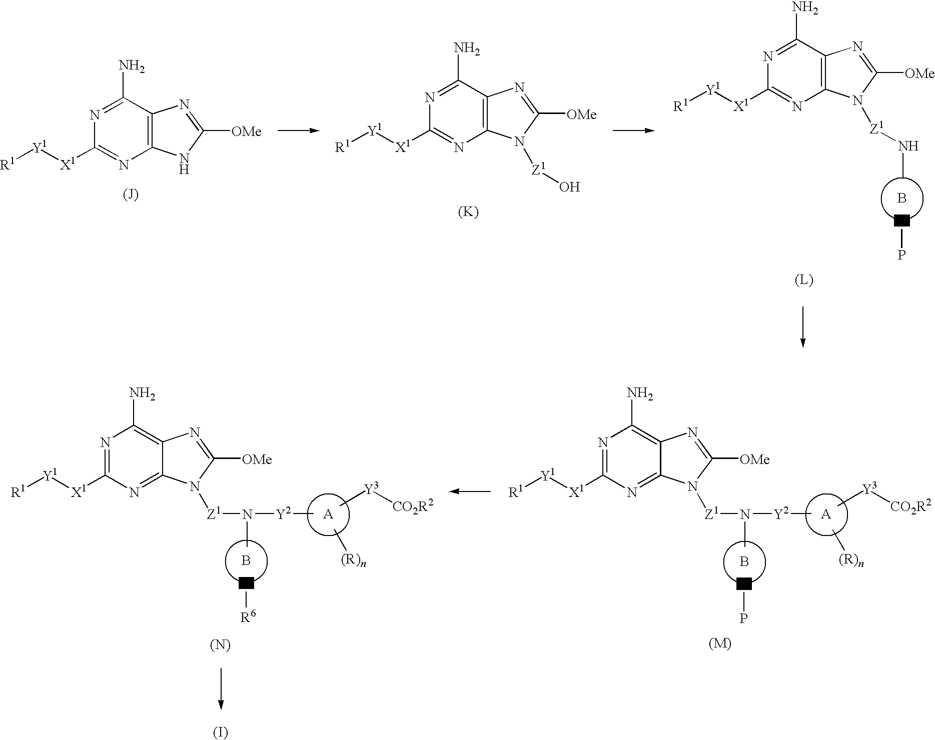

Compounds of formula (K) may be prepared by reacting the compound of formula (J) with a compound of formula (XIX);

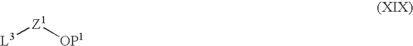

wherein L³ represents a leaving group such as a halogen, mesylate or triflate, Z¹ is as defined in formula (I) and P1 is an oxygen protecting group such as acetate or silyl. The reaction may be carried out in an organic solvent such as N,N-dimethylformamide, dimethylsulfoxide or acetonitrile with a base present, preferably at room temperature (20° C.). A base such as an alkali metal carbonate, e.g. sodium carbonate or potassium carbonate; an alkaline earth metal carbonate, e.g. calcium carbonate; a metal hydroxide, e.g. sodium hydroxide or potassium hydroxide; a metal hydrogenate, e.g. sodium hydride; or a metal alkoxide, e.g. potassium t-butoxide, may be used. The oxygen protecting group is then removed to provide the alcohol.

A compound of formula (L) can be prepared by a standard Mitsunobu reaction between a compound of formula (K) and a compound of formula (XX) followed by removal of the nosylate group;

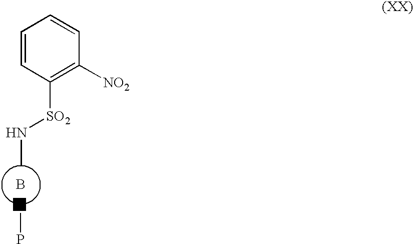

The nosylate group may be removed using 2-mercaptoethanol and a base such as potassium carbonate in DMF at elevated temperatures.

Compounds of formula (M) may be prepared by treating a compound of formula (L) with a compound of formula (V) or (VI) under similar conditions as described before.

Compounds of formula (N) may be prepared by treating a compound of formula (M) by deprotection of the nitrogen protecting group, followed by reaction with an appropriate aldehyde or ketone in the presence of a reducing agent such as sodium triacetoxyborohydride or sodium cyano borohydride.

A compound of formula (I) may be obtained from a compound of formula (N) by deprotection of the methyl group using HCl in methanol.

Compounds of formulae (III), (V), (VI), (VIII), (X), (XII), (XIII), (XVI), (XVII), (XVIII) and (XX) are either commercially available, are known in the literature or may be prepared using known techniques. Novel intermediates form a further aspect of the invention.

Compounds of formula (I) can be converted into further compounds of formula (I) using standard procedures. For example a compound of formula (I) where R²=methyl can be converted to a compound of formula (I) where R²=ethyl by treatment with a solution of hydrogen chloride in ethanol, at a temperature, for example in the range from 20 to 78° C.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of toll-like receptor (especially TLR7) activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention may be used in the treatment of asthma, COPD, allergic rhinitis, allergic conjunctivitis, atopic dermatitis, cancer, hepatitis B, hepatitis C, HIV, HPV, bacterial infections and dermatosis.

The invention still further provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% ow, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention (including pharmaceutically acceptable salts) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases COPD, asthma and allergic rhinitis the compounds of the invention may be combined with agents such as tumour necrosis factor alpha (TNF-alpha) inhibitors such as anti-TNF monoclonal antibodies (for example Remicade, CDP-870 and adalimumab) and TNF receptor immunoglobulin molecules (such as Enbrel); non-selective cyclo-oxygenase COX-1/COX-2 inhibitors whether applied topically or systemically (such as piroxicam, diclofenac, propionic acids such as naproxen, flubiprofen, fenoprofen, ketoprofen and ibuprofen, fenamates such as mefenamic acid, indomethacin, sulindac, azapropazone, pyrazolones such as phenylbutazone, salicylates such as aspirin), COX-2 inhibitors (such as meloxicam, celecoxib, rofecoxib, valdecoxib, lumarocoxib, parecoxib and etoricoxib); glucocorticosteroids (whether administered by topical, oral, intramuscular, intravenous, or intra-articular routes); methotrexate, lefumomide; hydroxychloroquine, d-penicillamine, auranofin or other parenteral or oral gold preparations.

The present invention still further relates to the combination of a compound of the invention and a leulcotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT B4, LTC4, LTD4, and LTE4) selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agent including muscarinic receptor (M1, M2, and M3) antagonists such as atropine, hyoscine, glycopyrrrolate, ipratropium bromide, tiotropium bromide, oxitropium bromide, pirenzepine or telenzepine.

The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol.

The present invention further relates to the combination of a compound of the invention and a chromone, such as sodium cromoglycate or nedocromil sodium.

The present invention still further relates to the combination of a compound of the invention together with an insulin-like growth factor type I (IGF-1) mimetic.

The present invention still further relates to the combination of a compound of the invention and a glucocorticoid, such as flunisolide, triamcinolone acetonide, beclomethasone dipropionate, budesonide, fluticasone propionate, ciclesonide or mometasone furoate.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-1) and MMP-9 and MMP-12.

The present invention still further relates to the combination of a compound of the invention together with modulators of chemokine receptor function such as antagonists of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX3CR1 for the C—X3-C family.

The present invention still further relates to the combination of a compound of the invention together with a cytokine or modulator of cytokine function, including alpha-, beta-, and gamma-interferon; interleukins (IL) including IL1 to 15, and interleukin antagonists or inhibitors, including agents which act on cytokine signalling pathways.

The present invention still further relates to the combination of a compound of the invention together with an immunoglobulin (Ig) or Ig preparation or an antagonist or antibody modulating Ig function such as anti-IgE (omalizumab).

The present invention further relates to the combination of a compound of the invention and another systemic or topically-applied anti-inflammatory agent, such as thalidomide or a derivative thereof, a retinoid, dithranol or calcipotriol.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example falvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will be further explained by reference to the following illustrative examples.

The following abbreviations are used;
EtOAc ethyl acetate
DCM dichloromethane
NMP N-methylpyrrolidine
NBS N-bromosuccinamide
DMF N,N-dimethylformamide
DMSO dimethylsulfoxide
THF tetrahydrofuran
TFA trifluoroacetic acid
mcpba 3-chloroperoxybenzoic acid (Aldrich 77% max)
   rt room temperature
   h hours
min minutes
M molar
MS mass spectrometry
APCI atmospheric pressure chemical ionisation
NMR nuclear magnetic resonance
HCl hydrochloric acid
BOC tertiary-butoxycarbonyl
HOBt 1-hydroxybenzotriazole
EDC 1-(3-dimethylamino propyl)-3-ethylcarbodiimide hydrochloride
HATU O-(7-azabenzotriazol-1-yl)-N,N,N′,N′-tetramethyluronium hexafluorophosphonate

Unless otherwise stated organic solutions were dried over magnesium sulphate. RPHPLC denotes Reverse Phase Preparative High Performance Liquid Chromatography using Waters Symmetry C8, Xterra or Phenomenex Gemini columns using acetonitrile and either aqueous ammonium acetate, ammonia, formic acid or trifluoroacetic acid as buffer where appropriate. Column chromatography was carried out on silica gel. SCX denotes solid phase extraction with a sulfonic acid sorbent whereby a mixture was absorbed on a sulfonic acid sorbent and eluted with an appropriate solvent such as methanol or acetonitrile and then the free base product was eluted with aqueous ammonia/an appropriate solvent such as methanol or acetonitrile.

### EXAMPLE 1

### Methyl (3-{[[3-(6-amino-2-butoxy-8-oxo-7,8-dihydro-9H-purin-9-yl)propyl] (piperidin-4-yl)amino]methyl}phenyl)acetate

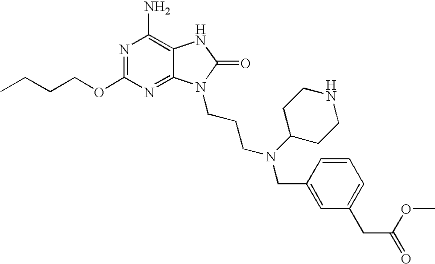

### (i) 2-Chloro-9-(tetrahydro-2H-pyran-2-yl)-9H-purin-6-amine

2,6-Dichloro-9-(tetrahydro-2H-pyran-2-yl)-9H-purine (55 g) was dissolved in 7N-aqueous ammonia in methanol (500 ml) and heated at 100° C. in a sealed flask for 6 h. The reaction mixture was cooled to rt and left overnight. Filtration afforded the subtitle compound, yield 40 g.

¹H NMR δ (CDCl₃) 8.02 (1H, s), 5.94 (2H, brs), 5.71 (1H, dd), 4.15-4.22 (1H, m), 3.75-3.82 (1H, m), 1.27-2.12 (6H, m).

### (ii) 2-Butoxy-9-(tetrahydro-2H-pyran-2-yl)-9H-purin-6-amine

The product from step (i) (40 g) was dissolved in 19% (w/w)-sodium n-butoxide in butanol (250 ml). The reaction mixture was stirred under reflux for 6 h. The resultant suspension was cooled to rt, diluted with water and extracted with diethyl ether. The combined organic phase was washed with water and dried and concentrated in vacuo. The subtitle compound was crystallized from diethyl ether/isohexane and obtained by filtration, yield 19 g.

¹H NMR δ (CDCl₃) 7.87 (1H, s), 5.56-5.68 (3H, m), 4.31-4.35 (2H, t), 4.14-4.17 (1H, m), 3.76-3.80 (1H, m), 1.49-2.08 (10H, m), 0.98 (3H, t).

### (iii) 8-Bromo-2-butoxy-9-(tetrahydro-2H-pyran-2-yl) 9H-purin-6-amine

The product from step (ii) (30 g) was dissolved in dry DCM (200 ml). The solution was stirred at rt, whilst NBS (27 g) was added portionwise. The mixture was stirred at rt overnight, then 20% (w/v)-sodium sulfate was added and the separated aqueous phase extracted with DCM. The combined organic phase was washed with saturated sodium hydrogen carbonate solution and brine. After concentration in vacuo, the residue was dissolved in EtOAc, washed with water and brine, and dried. The solution was filtered through silica gel and concentrated in vacuo. The residue was triturated with diethyl ether and isohexane, then filtered to give the subtitle compound (26 g). The filtrate was concentrated in vacuo and the residue purified by column chromatography (EtOAc/isohexane), to give a further 2.5 g of product. The solids were combined to give the subtitle compound as a yellow solid, yield 28.5 g.

¹H NMR δ (CDCl₃) 5.59-5.64 (3H, m), 4.32 (2H, m), 4.17 (1H, m), 3.74 (1H, m), 3.08 (1H, m), 2.13 (1H, d), 1.48-1.83 (8H, m), 0.98 (3H, t). mp 148-50° C.

### (iv) 2-Butoxy-8-methoxy-9-(tetrahydro-2H-pyran-2-yl) 9H-purin-6-amine

Sodium (3.7 g) was added to absolute methanol (400 ml) under a nitrogen atmosphere. To this solution was added the product (28.5 g) from step (iii) and the mixture stirred at 65° C. for 9 h. The mixture was concentrated in vacuo, then water added. The aqueous phase was extracted with EtOAc, washed with brine and dried. The subtitle compound was obtained after crystallisation from diethyl ether, yield 14.2 g.

¹H NMR δ (CDCl₃) 5.51 (1H, dd), 5.28 (2H, brs), 4.29 (2H, t), 4.11-4.14 (4H, m), 3.70 (1H, m), 2.76-2.80 (1H, m), 2.05 (1H, d), 1.47-1.81 (8H, m), 0.97 (3H, t).

### (v) 2-Butoxy-8-methoxy-9H-purin-6-amine, TFA salt

The product from step (iv) (24 g) was dissolved in absolute methanol (300 ml) and then TFA (30 ml) added. The reaction mixture was stirred at rt for 3 days and concentrated in vacuo. The subtitle compound was obtained as a white crystalline solid after trituration with methanol/EtOAc, yield 21 g.

¹H NMR δ (CD₃OD) 4.48 (2H, t), 4.15 (3H, s), 1.80 (2H, quintet), 1.50 (2H, sextet), 0.99 (3H, t).

### (vi) tert-Butyl [3-(6-amino-2-butoxy-8-methoxy-9H-purin-9-yl)propyl]carbamate

The product of step (v) (1.48 g), potassium carbonate (1.38 g) and tert-butyl (3-bromopropyl)carbamate (1.00 g) in dry DMF (10 ml) was stirred at 50° C. for 3 h, then cooled to rt. Water was added and the mixture extracted with EtOAc, washed with brine, dried and concentrated in vacuo. The residue was purified by column chromatography, to afford the subtitle compound, yield 1.10 g.

¹H NMR δ (DMSO-d₆) 6.82 (1H, t), 6.77 (2H, s), 4.17 (2H, t), 4.04 (3H, s), 3.83 (2H, t), 2.90 (2H, m), 1.79 (2H, m), 1.65 (2H, m), 1.41 (2H, m), 1.37 (9H, s), 0.92 (3H, t).

MS: APCI (+ve): 395 (M+H)

### (vii) 6-Amino-9-(3-aminopropyl)-2-butoxy-7,9-dihydro-8H-purin-8-one

The product of step (vi) (1.1 g) was dissolved in methanol/DCM (40 ml, 1/1), 4M−HCl in dioxane (10 ml) added and stirred at rt for 20 h. The mixture was concentrated in vacuo and the residue treated with SCX, to give the subtitle compound as a solid, yield 0.70 g.

¹H NMR δ (DMSO-d₆) 6.41 (2H, s), 4.14 (2H, t), 3.72 (2H, t), 3.37-3.26 (3H, m), 2.48 (2H, m), 1.67 (4H, m), 1.39 (2H, m), 0.92 (3H, t).

MS: APCI (+ve): 281 (M+H)

### (viii) Methyl (3-{[[3-(6-amino-2-butoxy-8-oxo-7,8-dihydro-9H-purin-9-yl)propyl] (piperidin-4-yl)amino]methyl}phenyl)acetate

The product of step (vii) (0.50 g) and 4-oxo-piperidine-1-carboxylic acid tert-butyl ester (0.39 g) were stirred together with 3 drops of glacial acetic acid in NMP (20 ml) at rt for 5 min. Sodium triacetoxyborohydride (1.13 g) was added, and the solution stirred at 40° C. overnight. Methyl (3-formylphenyl)acetate (0.38 g) was added along with a further 1 g of sodium triacetoxyborohydride and the mixture stirred overnight. A further 0.2 g of methyl (3-formylphenyl)acetate was added and the mixture left at 40° C. for 24 h. The mixture was purified by SCX and the product dissolved in a mixture of DCM/TFA (3/1, 40 ml). After stirring at rt for 24 h, the mixture was concentrated in vacuo and the residue purified by RPHPLC, yield 0.50 g.

¹HNMR δ (DMSO-d₆) 7.26-7.17 (4H, m), 7.12-7.03 (2H, m), 6.38 (2H, s), 4.11 (3H, t), 3.66-3.60 (4H, m), 3.58 (3H, s), 3.55 (1H, s), 2.96-2.90 (2H, m), 2.50-2.42 (2H, m), 2.36-2.28 (2H, m), 1.78-1.70 (2H, m), 1.65-1.56 (4H, m), 1.40-1.34 (2H, m), 1.33-1.26 (2H, m), 0.90 (3H, t).

MS: APCI (+ve): 526 (M+H)

### EXAMPLE 2

### Methyl [3-({(1-acetylpiperidin-4-yl)[3-(6-amino-2-butoxy-8-oxo-7,8-dihydro-9H-purin-9-yl)propyl]amino}methyl)phenyl]acetate

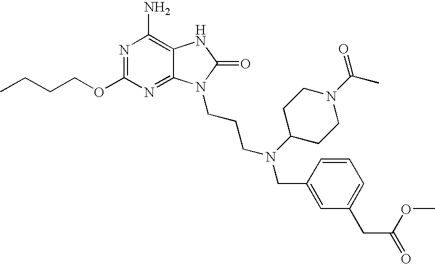

The product of example 1 (0.10 g) was dissolved in acetonitrile (2 ml) and treated with acetyl chloride (22 mg). The mixture was stirred at rt overnight then purified by RPHPLC, to afford the title compound as a white solid, yield 69 mg.

¹H NMR δ (MSO-d₆) 9.79 (1H, s), 7.24-7.18 (3H, m), 7.11-7.05 (1H, m), 6.37 (2H, s), 4.45-4.36 (1H, m), 4.11 (2H, t), 3.85-3.77 (1H, m), 3.66-3.61 (4H, m), 3.58 (3H, s), 3.56 (2H, s), 2.94-2.84 (1H, m), 2.73-2.63 (1H, m), 2.49-2.43 (2H, m), 2.41-2.31 (2H, m), 1.96 (3H, s), 1.78-1.71 (2H, m), 1.70-1.64 (2H, m), 1.64-1.58 (2H, m), 1.43-1.31 (2H, m), 1.27-1.19 (2H, m), 0.90 (1H, t).

MS: APCI (+ve): 568 (M+H)

### EXAMPLE 3

### Methyl [3-({[3-(6-amino-2-butoxy-8-oxo-7,8-dihydro-9H-purin-9-yl)propyl][1-(2-hydroxyethyl)piperidin-4-yl]amino}methyl)phenyl]acetate

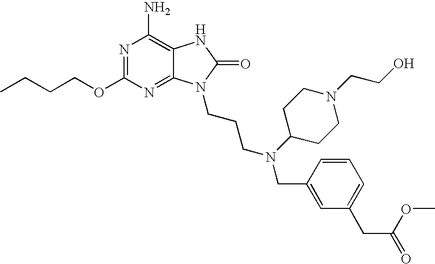

The product of example 1 (0.10 g) was dissolved in NMP (3 ml), treated with triethylamine (0.23 g) and 2-bromoethanol (0.04 g) added. The reaction mixture was stirred at rt for 16 h. The mixture was purified via RPHPLC, to afford the title compound, yield 32 mg.

¹H NMR δ (DMSO-d₆) 7.75-7.64 (3H, m), 7.60-7.51 (1H, m), 6.89-6.82 (2H, m), 4.64-4.55 (2H, m), 4.15-4.09 (4H, m), 4.06 (3H, s), 4.05-4.00 (2H, m), 3.94-3.85 (1H, m), 3.82-3.72 (6H, m), 3.45-3.32 (2H, m), 2.83-2.75 (2H, m), 2.26-2.17 (2H, m), 2.13-2.04 (4H, m), 1.89-1.80 (2H, m), 1.79-1.70 (2H, m), 1.42-1.35 (3H, m).

MS: APCI (+ve): 570 (M+H)

### EXAMPLE 4

### Methyl [3-({[3-(6-amino-2-butoxy-8-oxo-7,8-dihydro-9H-purin-9-yl)propyl][1-(3-hydroxypropyl)piperidin-4-yl]amino}methyl)phenyl]acetate

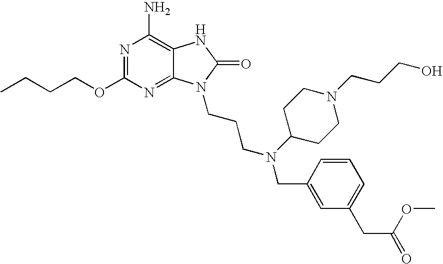

The title compound was prepared by the method of example 3 using the product from the example 1 and 3-bromopropanol, yield 49 mg.

¹H NMR δ (DMSO-d₆) 7.24-7.17 (4H, m), 7.10-7.05 (1H, m), 6.37 (2H, s), 4.11 (2H, t), 3.66-3.60 (4H, m), 3.58 (3H, s), 3.56-3.55 (2H, m), 3.40 (2H, t), 2.97-2.89 (1H, m), 2.88-2.80 (1H, m), 2.49-2.43 (2H, m), 2.35-2.21 (4H, m), 1.79-1.69 (2H, m), 1.67-1.57 (4H, m), 1.57-1.47 (2H, m), 1.42-1.28 (4H, m), 0.90 (3H, t).

MS: APCI (+ve): 584 (M+H)

### EXAMPLE 5

### Methyl (3-{[[3-(6-amino-2-butoxy-8-oxo-7,8-dihydro-9H-purin-9-yl)propyl](1-methylpiperidin-4-yl)amino]methyl}phenyl)acetate

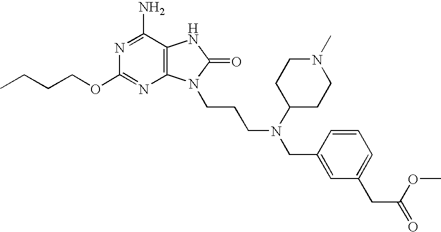

The product of example 1 step (vii) (430 mg), N-methylpiperidone (191 mg), sodium triacetoxyborohydride (1.1 g) and acetic acid (0.5 ml) were stirred together in NMP (10 ml) at 50° C. for 2 h. The mixture was cooled to rt and treated with SCX. After concentration in vacuo, the residue was dissolved in NMP (10 ml) and methyl (3-formylphenyl)acetate (222 mg), sodium triacetoxyborohydride 1.1 g and a few drops of acetic acid added. The mixture was stirred at 45° C. for 24 h. The mixture was cooled to rt, treated with SCX and purified by RPHPLC, to afford the title compound, yield 370 mg.

¹H NMR δ (CDCl₃) 7.22-7.05 (4H, m), 5.39 (2H, s), 4.25 (2H, t), 3.81 (2H, t), 3.71 (3H, s), 3.64-3.61 (2H, m), 3.59-3.56 (2H, m), 2.91-2.83 (2H, m), 2.59-2.43 (3H, m), 2.23 (3H, s), 1.98-1.19 (12H, m), 1.00-0.92 (3H, m).

MS: APCI (+ve): 540 (M+H)

### EXAMPLE 6

### Methyl (3-{[[3-(6-amino-2-butoxy-8-oxo-7,8-dihydro-9H-purin-9-yl)propyl] (1-ethylpiperidin-4-yl)amino]methyl}phenyl)acetate

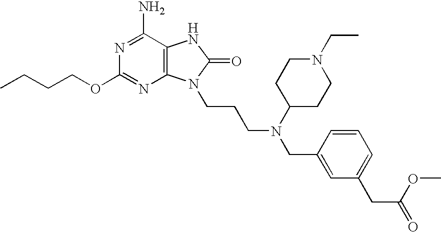

The title compound was prepared by the method of example 5 using N-ethylpiperidone, yield 50 mg.

¹H NMR δ (DMSO-d₆) 9.82 (1H, s), 7.21-7.17 (3H, m), 7.09-7.04 (1H, m), 6.37 (2H, s), 4.11 (2H, t), 3.66-3.59 (4H, m), 3.57 (2H, s), 3.54 (2H, s), 2.84 (2H, d), 2.52-2.32 (4H, m), 2.22 (2H, q), 1.79-1.54 (7H, m), 1.48-1.28 (5H, m), 0.97-0.84 (6H, m).

MS: APCI (+ve): 554 (M+H)

### EXAMPLE 7

### Methyl [3-({[3-(6-amino-2-butoxy-8-oxo-7,8-dihydro-9H-purin-9-yl)propyl][1-(2-tert-butoxy-2-oxoethyl)piperidin-4-yl]amino}methyl)phenyl]acetate

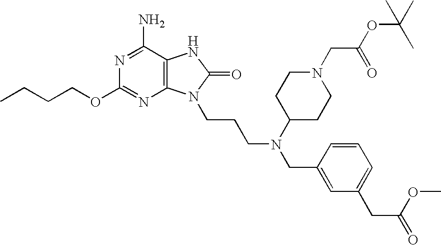

The title compound was prepared by the method of example 5 using tert-butyl(4-aminopiperidin-1-yl)acetate, yield 340 mg.

¹H NMR δ (CDCl₃) 9.97 (1H, s), 7.24-7.14 (3H, m), 7.11-7.06 (2H, m), 5.46 (2H, s), 4.25 (2H, t), 3.81 (2H, t), 3.70 (3H, s), 3.62 (2H, s), 3.58 (2H, s), 3.06 (2H, s), 3.00-2.92 (2H, m), 2.58-2.44 (2H, m), 2.14-2.02 (2H, m), 1.93-1.81 (2H, m), 1.79-1.65 (4H, m), 1.53-1.46 (4H, m), 1.45 (9H, s), 0.96 (3H, t).

MS: APCI (+ve): 640 (M+H)

### EXAMPLE 8

### (4-{[3-(6-Amino-2-butoxy-8-oxo-7,8-dihydro-9H-purin-9-yl)propyl][3-(2-methoxy-2-oxoethyl)benzyl]amino}piperidin-1-yl)acetic acid

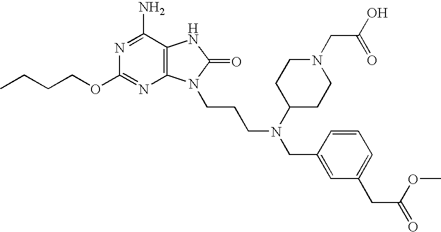

The product from example 7 was dissolved in a mixture of DCM/TFA (5/1, 18 ml) and stirred at rt for 72 h. The mixture was concentrated inz vacuo and the residue purified by RPHPLC, yield 230 mg.

¹H NMR δ (DMSO-d₆) 9.97 (1H, s), 7.26-7.21 (3H, m), 7.15-7.07 (1H, m), 6.45 (2H, s), 4.13 (2H, t), 3.70-3.63 (4H, m), 3.60 (5H, s), 3.28-3.18 (4H, m), 1.86-1.53 (12H, m), 1.45-1.30 (3H, m), 0.92 (3H, t).

MS: APCI (−ve): 582 (M−H)

### EXAMPLE 9

### Methyl (3-{[[3-(6-amino-2-butoxy-8-oxo-7,8-dihydro-9H-purin-9-yl)propyl] (1-benzylpiperidin-4-yl)amino]methyl}phenyl)acetate

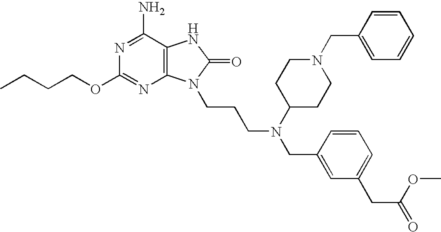

### (i) 9-(3-Bromopropyl)-2-butoxy-8-methoxy-9H-purin-6-amine

The product of example 1 step (v) (20 g) was added in portions over 10 min to a rapidly stirred mixture of potassium carbonate (40 g) and 1,3-dibromopropane (34 ml) in DMF (250 ml) at rt and the mixture stirred for 1.5 h. The mixture was diluted with water and extracted with EtOAc. The combined extracts were washed with brine and dried. The mixture was purified by column chromatography, to afford the subtitle compound as a white solid, yield 16 g.

¹H NMR δ (CDCl₃) 5.19 (2H, s), 4.28 (2H, t), 4.12 (3H, s), 4.09 (2H, t), 3.37 (2H, t), 2.39-2.30 (2H, m), 1.81-1.72 (2H, m), 1.55-1.43 (2H, m), 0.96 (3H, J=11.4 Hz, t).

### (ii) 6-Amino-9-(3-bromopropyl)-2-butoxy-7,9-dihydro-8H-purin-8-one, hydrochloride

The product of step (i) (35.8 g) was dissolved in methanol (400 ml) and treated with 4M−HCl in dioxane (100 ml). The mixture was stirred at rt for 6 h and concentrated in vacuo. DCM was added, and the solution concentrated in vacuo, to afford a subtitle compound as a foam, which was then taken onto the next step without further purification, yield 38 g.

¹H NMR δ (DMSO-d₆) 10.60 (1H, s), 4.45 (2H, m), 3.84 (2H, m), 3.65 (2H, m), 2.19 (2H, m), 1.66-1.73 (2H, m), 1.36-1.47 (2H, m), 0.96 (3H, m).

### (iii) 6-Amino-9-{3-[(1-benzylpiperidin-4-yl)amino]propyl}-2-butoxy-7,9-dihydro-8H-purin-8-one

A solution of the product from step (ii) (1.0 g) and 1-benzylpiperidin-4-amine (5 ml) in acetonitrile (10 ml) was heated at 80° C. for 12 h. The solvent was removed under reduced pressure and the residue purified by RPHPLC, yield 400 mg.

MS: APCI (+ve): 454 (+H)

### (iv) Methyl (3-{[[3-(6-amino-2-butoxy-8-oxo-7,8-dihydro-9H-purin-9-yl)propyl](1-benzylpiperidin-4-yl)amino]methyl}phenyl)acetate

A mixture of the product from step (iii) (0.34 g), methyl (3-formylphenyl)acetate (150 mg), sodium triacetoxyborohydride (652 mg) and acetic acid (0.5 ml) in NMP (10 ml) were stirred together at 45° C. for 24 h. The mixture was cooled to rt, treated with SCX and purified by RPHPLC, to afford the title compound, yield 240 mg.

¹H NMR δ (DMSO-d₆) 9.80 (1H, s), 7.38-7.16 (8H, m), 7.13-7.07 (1H, m), 6.38 (2H, s), 4.13 (2H, t), 3.69-3.61 (4H, m), 3.59 (3H, s), 3.57 (2H, s), 3.42 (3H, s), 3.31 (2H, s), 2.88-2.78 (2H, m), 1.90-1.21 (12H, m), 0.91 (3H, t).

MS: APCI (+ve): 616 (M+H)

### EXAMPLE 10

### Methyl (3-{[[4-(6-amino-2-butoxy-8-oxo-7,8-dihydro-9H-purin-9-yl)butyl] (1-methylpiperidin-4-yl)amino]methyl}phenyl)acetate

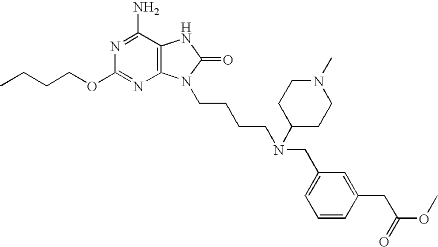

### (i) 9-(4-Bromobutyl)-2-butoxy-8-methoxy-9H-purin-6-amine

The subtitle compound was prepared by the method of example 9 step (i) using 1,4-dibromobutane, yield 16 g.

¹H NMR δ (DMSO-d₆) 6.77 (2H, s), 4.17 (2H, t), 4.05 (3H, s), 3.86 (2H, t), 3.55 (2H, t), 1.85-1.69 (6H, m), 1.68-1.60 (2H, m), 1.44-1.34 (2H, m), 0.91 (3H, t)

### (ii) 2-Butoxy-8-methoxy-9-{4-[(1-methylpiperidin-4-yl)amino]butyl}-9H-purin-6-amine

The product of step (i) (1.0 g) and 1-methylpiperidin-4-amine (3.3 g) were stirred together in acetonitrile at 80° C. for 2 h. After cooling to rt, the mixture was purified by RPHPLC, to afford the subtitle compound as a cream solid, yield 520 mg.

MS: APCI (+ve): 406 (M+H)

### (iii) Methyl (3-{[[4-(6-amino-2-butoxy-8-oxo-7,8-dihydro-9H-purin-9-yl)butyl](1-methylpiperidin-4-yl)amino]methyl}phenyl)acetate

The product of step (ii) (560 mg), methyl (3-formylphenyl)acetate (286 mg) and sodium triacetoxyborohydride (922 mg) were stirred together in NMP (20 ml) at 50° C. for 24 h. The mixture was cooled to rt, treated with SCX and purified by RPHPLC. Methanol (5 ml) and 4M−HCl in dioxane (1 ml) were added and stirred at rt overnight. The mixture was concentrated in vacuo, to afford the title compound, yield 130 mg.

¹H NMR δ (DMSO-d₆) 9.84 (1H, s), 7.22-7.10 (3H, m), 7.08-7.03 (1H, m), 6.38 (2H, s), 4.12 (2H, t), 3.66-3.54 (5H, m), 3.51 (2H, s), 3.32 (3H, s), 2.73 (2H, d), 2.46-2.36 (2H, m), 2.35-2.25 (2H, m), 2.08 (3H, s), 1.72-1.17 (12H, m), 0.90 (3H, t).

MS: APCI (+ve): 554 (M+H)

### EXAMPLE 11

### Methyl (3-{2-[[3-(6-amino-2-butoxy-8-oxo-7,8-dihydro-9H-purin-9-yl)propyl](1-methylpiperidin-4-yl)amino]-2-oxo ethyl}phenyl)acetate

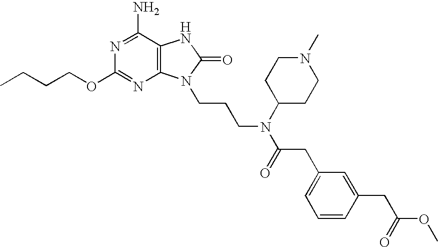

### (i) 6-Amino-2-butoxy-9-{3-[(1-methylpiperidin-4-yl)amino]propyl}-7,9-dihydro-8H-purin-8-one

The product of example 9 step (ii) (1.0 g) was suspended in acetonitrile (100 ml) and 1-methylpiperidine-4-amine (3.3 g) added. The mixture was stirred under reflux overnight. After cooling to rt, the mixture was concentrated in vacuo and purified by RPHPLC, to afford the subtitle compound as a cream solid, yield 1 g.

MS: APCI (+ve): 378 (M+H)

### (ii) Methyl (3-{2-[[3-(6-amino-2-butoxy-8-oxo-7,8-dihydro-9H-purin-9-yl)propyl](1-methylpiperidin-4-yl)amino]-2-oxoethyl}phenyl)acetate

The product of step (i) (200 mg) was dissolved in DMF (5 ml), then EDC (203 mg), HOBt (143 mg) and [3-(2-methoxy-2-oxoethyl)phenyl]acetic acid (221 mg) were added. The mixture was stirred at rt overnight, treated with SCX and purified by RPHPLC, to afford the title compound as a white solid, yield 1 mg.

¹H NMR δ (DMSO-d₆) 9.93 (1H, brs), 7.24-6.98 (4H, m), 6.46-6.42 (2H, brs), 4.15 (2H, m), 3.71-3.53 (9H, m), 3.14 (2H, m), 2.78-2.64 (2H, m), 2.12 (3H, brs), 1.84-1.21 (13H, m), 0.88 (3H, m).

MS: APCI (+ve): 568 (M+H)

### EXAMPLE 12

### Methyl [3-({[3-(6-amino-2-butoxy-8-oxo-7,8-dihydro-9H-purin-9-yl)propyl][(3R)-1-benzylpyrrolidin-3-yl]amino}methyl)phenyl]acetate

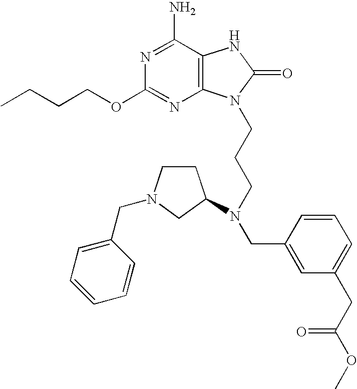

The title compound was prepared by the method of example 9 step (iii) and (iv) using the product from the example 9 step (ii) and (3R)-(−)-1-benzyl-3-aminopyrrolidine, yield 0.28 g.

¹H NMR δ (DMSO-d₆) 9.82 (1H, s), 7.33-7.06 (9H, m), 6.39 (2H, s), 4.13 (2H, t), 3.64 (2H, s), 3.60 (3H, s), 3.57-3.42 (6H, m), 3.31 (2H, s), 2.47-2.23 (7H, m), 1.89-1.72 (2H, m), 1.69-1.57 (2H, m), 1.45-1.31 (2H, m), 0.91 (3H, t).

MS: APCI (+ve): 602 (M+H)

### EXAMPLE 13

### Methyl (3-{[[3-(6-amino-2-butoxy-8-oxo-7,8-dihydro-9H-purin-9-yl)propyl] (1-isopropylpiperidin-4-yl)amino]methyl}phenyl)acetate

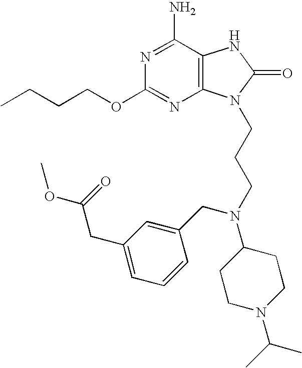

### (i) 6-Amino-2-butoxy-9-{3-[(1-isopropylpiperidin-4-yl)amino]propyl}-7,9-dihydro-8H-purin-8-one

The product from example 1 step (vii) (diHCl salt) (0.8 g) and triethylamine (0.4 ml) in NMP (10 ml) were stirred together at rt for 1 h. Sodium triacetoxyborohydride (1.0 g), acetic acid (1 ml) and 1-isopropyl-4-piperidone (0.22 g) were added and the mixture stirred at 40° C. for 24 h. The mixture was cooled to rt, treated with SCX and purified by RPHPLC, yield 295 mg.

### (ii) Methyl (3-{[[3-(6-amino-2-butoxy-8-oxo-7,8-dihydro-9H-purin-9-yl)propyl](1-isopropylpiperidin-4-yl)amino]methyl}phenyl)acetate

The title compound was prepared by the method of example 9 step (iv) using the compound from step (i), yield 0.16 g

¹H NMR δ (DMSO-d₆) 7.25-7.20 (3H, m), 7.13-7.06 (1H, m), 6.41 (2H, s), 4.14 (2H, t), 3.65 (2H, s), 3.60 (3H, s), 3.56 (2H, s), 2.83-2.73 (2H, m), 2.70-2.58 (2H, m), 2.49-2.40 (2H, m), 2.05-1.91 (2H, m), 1.83-1.56 (6H, m), 1.47-1.31 (6H, m), 0.98-0.88 (9H, m).

MS: APCI (+ve): 568 (M+H)

### EXAMPLE 14

### Methyl [3-({[3-(6-amino-2-butoxy-8-oxo-7,8-dihydro-9H-purin-9-yl)propyl][1-(cyanomethyl)piperidin-4-yl]amino}methyl)phenyl]acetate

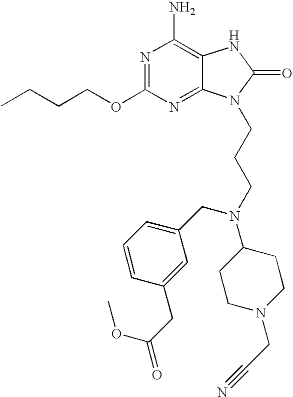

The title compound was prepared by the method of example 3 using the compound from example 1 and bromoacetonitrile, yield 195 mg.

¹H NMR δ (DMSO-d₆) 9.79 (1H, s), 7.30-7.13 (3H, m), 7.12-7.04 (1H, m), 6.36 (2H, s), 4.12 (2H, t), 3.67-3.63 (4H, m), 3.58 (3H, s), 3.56 (2H, s), 2.82-2.76 (5H, m), 2.49-2.43 (2H, m), 2.09-2.00 (2H, m), 1.79-1.69 (2H, m), 1.67-1.57 (4H, m), 1.51-1.43 (2H, m), 1.40-1.33 (2H, m), 0.90 (3H, t).

MS: APCI (+ve): 565 (M+H)

### EXAMPLE 15

### Methyl [3-({[3-(6-amino-2-butoxy-8-oxo-7,8-dihydro-9H-purin-9-yl)propyl][1-(2-cyanoethyl)piperidin-4-yl]amino}methyl)phenyl]acetate

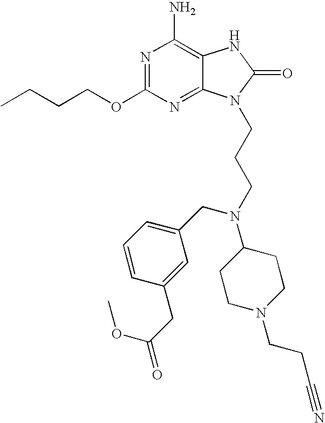

The title compound was prepared by the method of example 3 using the compound from example 1 and 3-bromopropionitrile, yield 75 mg.

¹H NMR δ (DMSO-d₆) 9.79 (1H, s), 7.23-7.16 (3H, m), 7.11-7.04 (1H, m), 6.36 (2H, s), 4.11 (2H, t), 3.68-3.60 (4H, m), 3.58 (3H, s), 3.55 (2H, s), 2.91-2.84 (2H, m), 2.67-2.43 (5H, m), 1.91-1.82 (2H, m), 1.77-1.69 (2H, m), 1.65-1.57 (4H, m), 1.50-1.41 (2H, m), 1.41-1.32 (2H, m), 0.90 (3H, t).

MS: APCI (+ve): 579 (M+H)

### EXAMPLE 16

### Methyl [3-({[3-(6-amino-2-butoxy-8-oxo-7,8-dihydro-9H-purin-9-yl)propyl][1-(3-cyanopropyl)piperidin-4-yl]amino}methyl)phenyl]acetate

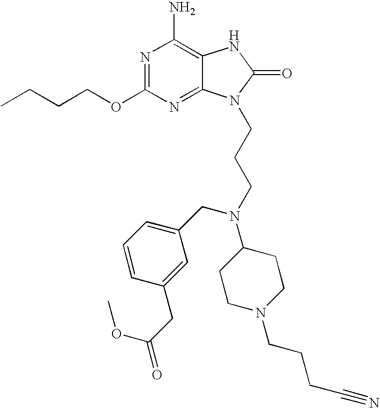

The title compound was prepared by the method of example 3 using the compound from example 1 and 4-bromo-butyronitrile, yield 85 mg.

¹H NMR δ (DMSO-d₆) 9.79 (1H, s), 7.22-7.18 (3H, m), 7.09-7.05 (1H, m), 6.36 (2H, s), 4.12 (2H, t), 3.66-3.61 (4H, m), 3.58 (3H, s), 3.55 (2H, s), 2.87-2.79 (2H, m), 2.53-2.40 (5H, m), 2.28-2.25 (2H, m), 1.80-1.71 (4H, m), 1.71-1.63 (4H, m), 1.63-1.58 (2H, m), 1.47-1.40 (2H, m), 1.40-1.33 (2H, m), 0.90 (3H, t).

MS: APCI (+ve): 593 (M+H)

### EXAMPLE 17

### tert-Butyl 4-(4-{[3-(6-amino-2-butoxy-8-oxo-7,8-dihydro-9H-purin-9-yl)propyl][3-(2-methoxy-2-oxoethyl)benzyl]amino}piperidin-1-yl)butanoate

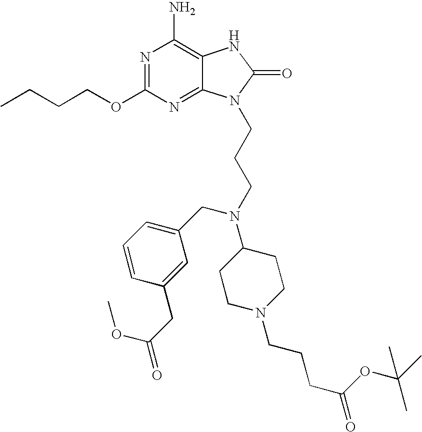

The title compound was prepared by the method of example 3 using the compound from example 1 and tert-butyl-4-bromobutyrate, yield 38 mg.

¹H NMR δ (DMSO-d₆) 9.79 (1H, s), 7.21-7.08 (4H, m), 6.36 (2H, s), 4.12 (2H, t), 3.63 (4H, m), 3.58 (3H, s), 3.54 (2H, s), 2.84-2.81 (2H, m), 2.52-2.30 (5H, m), 2.28-2.25 (2H, m), 2.19 (4H, m), 1.76-1.33 (21H, m), 0.90 (3H, t).

MS: APCI (+ve): 668 (M+H)

### EXAMPLE 18

### 4-(4-{[3-(6-Amino-2-butoxy-8-oxo-7,8-dihydro-9H-purin-9-yl)propyl][3-(2-methoxy-2-oxoethyl)benzyl]amino}piperidin-1-yl)butanoic acid

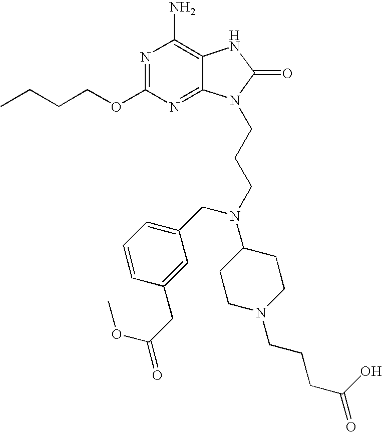

TFA (2 ml) was added to a solution of the product from example 17 (0.035 g) in DCM (4 ml) and the mixture stirred at rt for 3 h. The reaction mixture was concentrated in vacuo and the residue purified by RPHPLC, yield 3 mg.

¹H NMR δ (CDCl₃) 11.24 (1H, brs), 7.34-7.12 (4H, m), 6.27 (2H, brs), 4.10 (4H, m), 3.72-3.36 (9H, m), 2.84 (2H, m), 2.68-1.20 (23H, m), 0.88 (3H, m).

MS: APCI (+ve): 612 (M+H)

### EXAMPLE 19

### Methyl (3-{[{3-[6-amino-2-(2-methoxyethoxy)-8-oxo-7,8-dihydro-9H-purin-9-yl]propyl}(1-methylpiperidin-4-yl)amino]methyl}phenyl)acetate

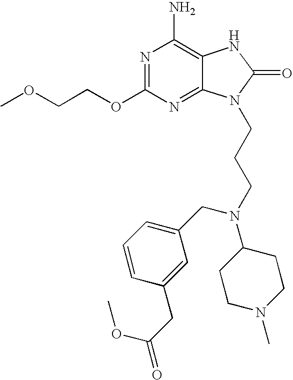

The title compound was prepared by the method of examples 1 using 2-methoxyethanol and example 5, yield 158 mg.

¹H NMR δ (DMSO-d₆) 9.83 (1H, brs), 7.24-7.21 (3H, m), 7.10-7.07 (1H, m), 6.41 (2H, s), 4.23 (2H, t), 3.64-3.62 (4H, m), 3.58-3.56 (5H, m), 3.55 (2H, s), 3.27 (3H, s), 2.76 (2H, d), 2.46 (2H, t), 2.40-2.33 (1H, m), 2.09 (3H, s), 1.76-1.70 (4H, m), 1.60 (2H, d), 1.48-1.40 (2H, m).

### EXAMPLE 20

### Methyl (4-{[[3-(6-amino-2-butoxy-8-oxo-7,8-dihydro-9H-purin-9-yl)propyl] (1-methylazetidin-3-yl)amino]methyl}phenyl)acetate

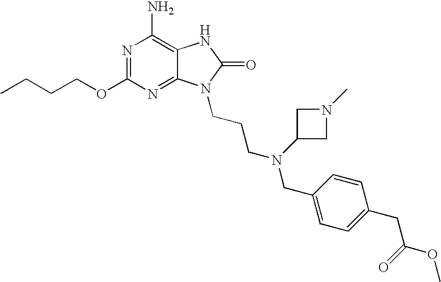

### (i) N-(tert-Butyloxycarbonylamino)-3-(2-nitrobenzenesulfonyl)aminoazetidine

To a solution of 3-amino-N-Boc-azetidine (1.98 g) in THF (40 ml) were added Et₃N (3.2 ml) and NsCl (2.43 g) at 0° C., and the mixture stirred at rt for 1 h. The reaction was quenched with brine, and extracted with EtOAc. The combined extracts were dried over MgSO₄ and concentrated, and the residue was crystallized from CHCl₃-Hexane to give the subtitle compound (3.94 g, 96%) as a white solid.

¹H NMR δ (CDCl₃) 8.13-8.07 (1H, m), 7.91-7.87 (1H, m), 7.80-7.73 (2H, m), 5.80 (1H, brd), 4.34-4.25 (1H, m), 4.09 (2H, brs), 3.76-3.69 (2H, m), 1.41 (9H, m).

### (ii) 2-Butoxy-9-(3-hydroxypropanyl)-8-methoxy-9H-purin-6-amine

To a suspension of the product from example 1 step (v) (50 g) in DMF (260 ml) was added K₂CO₃ (31 g), 3-acetoxy-3-bromopropane (31 g) and water (1.3 ml). After stirring at rt for 20 h, MeOH (198 g) and 0.5 N NaOH aq. (250 g) were added, and stirred at 80° C. for 4 h. The mixture was added dropwise to H₂O (500 ml), and cooled slowly to 4° C. The resulting suspension was filtered to afford the subtitle compound (30 g, 72%).

### (iii) tert-Butyl 3-{[3-(6-amino-2-butoxy-8-oxo-7,8-dihydro-9H-purin-9-yl)propyl][4-(2-methoxy-2-oxoethyl)benzyl]amino}azetidine-1-carboxylate

To a solution of the product from step (ii) (2.83 g) in THF (60 ml) were added the product from step (i) (3.94 g), PPh₃ (3.15 g) and DIAD (6.3 ml). The mixture was stirred at rt for 1 h, then concentrated. The residue was dissolved in DMF (60 ml), then 2-mercaptoethanol (0.88 ml) and K₂CO₃ (1.80 g) added. The reaction mixture was stirred at 60° C. for 3 h, quenched by satd. NaHCO₃ aq. and extracted with CHCl₃. The organic phase was dried over MgSO₄, concentrated and the residue was purified by flash silica gel column chromatography to afford the amine (4.21 g) as a white solid. To a solution of amine (4.21 g) in THF (90 ml) were added methyl (4-formylphenyl)acetate (2.00 g) and NaBH(OAc)₃ (2.98 g), and the mixture was stirred at rt for 26 h. The reaction was quenched by satd. Aq. NaHCO₃, and the mixture was extracted with CHCl₃. The organic extracts were dried over MgSO₄ and concentrated. The residue was purified by flash silica gel column chromatography to furnish the subtitle compound (5.12 g) as a colorless oil.

¹H NMR δ (DMSO-d₆) 7.20-7.14 (4H, m), 6.79 (2H, brs), 4.15 (2H, t), 4.03 (3H, s), 3.84 (2H, t), 3.77 (2H, brs), 3.65 (2H, s), 3.61 (3H, s), 3.62-3.54 (1H, m), 3.51-3.30 (6H, m), 2.33 (2H, t), 1.85 (2H, t), 1.68-1.60 (2H, m), 1.42-1.30 (2H, m), 1.36 (9H, s), 0.91 (3H, t).

### (iv) Methyl (4-{[[3-(6-amino-2-butoxy-8-oxo-7,8-dihydro-9H-purin-9-yl)propyl](1-methylazetidin-3-yl)amino]methyl}phenyl)acetate

The product from step (iii) (254 mg) was dissolved in TFA (10 ml), and the mixture was stirred at rt for 1 h. The solution was concentrated to give the TFA salt of amine. To a solution of the TFA salt in MeOH (5 ml) were added formaldehyde aq. (1 ml) and NaBH₃CN (130 mg) and the reaction mixture was stirred at rt for 1 h. The reaction was quenched by satd. NaHCO₃ aq. (10 ml), and the mixture was extracted with CHCl₃-MeOH (ca. 20:1) (50 ml×3). The combined extracts were dried over MgSO₄ and concentrated. The residue was purified by flash silica gel column chromatography to afford the amine (120 mg) as a colorless oil. To a solution of the amine (71.3 mg) in MeOH (4 ml) was added a solution of 4N HCl in 1,4-dioxane (4 ml). After stirring at rt for 5 h, the reaction was quenched by 28% NH₃ aq. (1 ml) and diluted with H₂O (15 ml). The mixture was extracted with CHCl₃-MeOH (ca. 20:1) (30 ml×3). The combined extracts were dried over MgSO₄ and concentrated to give the title compound (60.7 mg) as a white solid.

¹H NMR δ (DMSO-d₆) 9.87 (1H, brs), 7.24-7.18 (4H, m), 6.46 (2H, brs), 4.20 (2H, t), 3.74-3.68 (2H, m), 3.69 (2H, s), 3.66 (3H, s), 3.77 (2H, brs), 3.45 (2H, brs), 3.40-3.33 (2H, m), 3.18-3.12 (1H, m), 2.71-2.64 (2H, m), 2.32 (2H, brt), 2.21 (3H, s), 1.88-1.78 (2H, m), 1.72-1.65 (2H, m), 1.48-1.36 (2H, m), 0.96 (3H, t).

### EXAMPLE 21

### Methyl (4-{[[3-(6-amino-2-butoxy-8-oxo-7,8-dihydro-9H-purin-9-yl)propyl](1-ethylazetidin-3-yl)amino]methyl}phenyl)acetate

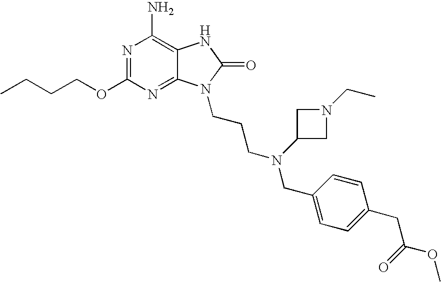

The title compound was prepared by the method of example 20 using acetaldehyde.

¹H NMR δ (DMSO-d₆) 9.76 (1H, brs), 7.12-7.06 (4H, m), 6.34 (2H, brs), 4.08 (2H, t), 3.62-3.56 (2H, m), 3.58 (2H, s), 3.54 (3H, s), 3.34 (2H, s), 3.22-3.18 (2H, m), 3.07-3.00 (2H, m), 2.52-2.45 (2H, m), 2.25-2.18 (4H, m), 1.76-1.68 (2H, m), 1.61-1.53 (2H, m), 1.36-1.26 (2H, m), 0.84 (3H, t), 0.75 (3H, t).

### EXAMPLE 22

### Methyl (4-{[[3-(6-amino-2-butoxy-8-oxo-7,8-dihydro-9H-purin-9-yl)propyl](1-isopropylazetidin-3-yl)amino]methyl}phenyl)acetate

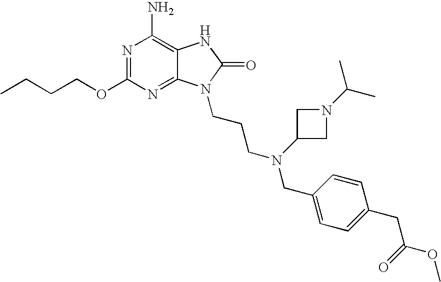

The title compound was prepared by the method of example 20 using acetone.

¹H NMR δ (DMSO-d₆) 9.83 (1H, brs), 7.23-7.13 (4H, m), 6.40 (2H, brs), 4.14 (2H, t), 3.67-3.64 (2H, m), 3.64 (2H, s), 3.61 (3H, s), 3.53-3.42 (3H, m), 3.24 (2H, brs), 3.03 (1H, brs), 2.25 (2H, brt), 1.85-1.74 (2H,), 1.67-1.61 (2H, m), 1.41-1.35 (2H, m), 0.94-0.74 (9H, m).

### EXAMPLE 23

### Methyl (4-{[[3-(6-amino-2-butoxy-8-oxo-7,8-dihydro-9H-purin-9-yl)propyl](1-isobutylazetidin-3-yl)amino]methyl}phenyl)acetate

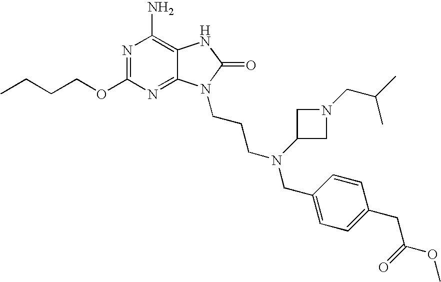

The title compound was prepared by the method of example 20 using isobutylaldehyde.

¹H NMR δ (DMSO-d₆) 9.82 (1H, brs), 7.18-7.12 (4H, m), 6.40 (2H, brs), 4.14 (2H, t), 3.66-3.61 (2H, m), 3.64 (2H, s), 3.60 (3H, s), 3.40 (2H, s), 3.30-3.22 (2H, m), 3.14-3.06 (2H, m), 2.60-2.54 (2H, m), 2.28-2.23 (2H, m), 2.10-2.05 (2H, m), 1.82-1.74 (2H, m), 1.67-1.59 (2H, m), 1.48-1.24 (5H, m), 0.91 (3H, t), 0.94-0.84 (1H, m), 0.78 (6H, d).

### EXAMPLE 24

### Methyl [4-({[3-(6-amino-2-butoxy-8-oxo-7,8-dihydro-9H-purin-9-yl)propyl][(3R)-1-methylpyrrolidin-3-yl]amino}methyl)phenyl]acetate

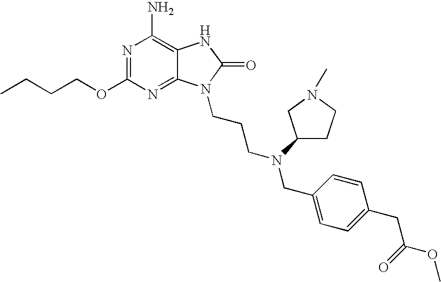

### (i) Benzyl (3R)-3-{[(2-nitrophenyl)sulfonyl]amino}pyrrolidine-1-carboxylate

To a solution of (3R)-(3-Boc-amino)pyrrolidine (758 mg) in CHCl₃ (30 ml) were added Et₃N (1.7 ml) and CbzCl (0.76 ml) at 0° C., and the mixture was stirred at rt for 1 h. The reaction was quenched by satd. NaHCO₃ aq., and extracted with CHCl₃. The combined extracts were dried over MgSO₄, concentrated, and the residue was purified by flash silica gel column chromatography to give the Cbz compound (1.27 g). The Cbz compound was dissolved in TFA (15 ml), and the mixture was stirred at rt for 30 min. The solution was concentrated to give the TFA salt of amine. To the solution of the TFA salt in THF (30 ml) were added Et₃N (3 ml) and NsCl (956 mg) and the reaction mixture was stirred at rt for 1 h. The reaction was quenched with satd. NaHCO₃ aq., and the mixture was extracted with CHCl₃. The combined extracts were dried over MgSO₄ and concentrated. The residue was purified by flash silica gel column chromatography to afford the subtitle compound (1.58 g) as a colorless oil.

¹H NMR δ (DMSO-d6) 8.57 (1H, brs), 8.02-7.95 (2H, m), 7.91-7.83 (2H, m), 7.40-7.28 (5H, m), 5.07-5.02 (2H, m), 3.89-3.80 (1H, m), 3.48-3.20 (3H, m), 3.19-3.09 (1H, m), 2.04-1.90 (1H, m), 1.85-1.74 (1H, m).

### (ii) Benzyl (3R)-3-{[3-(6-amino-2-butoxy-8-oxo-7,8-dihydro-9H-purin-9-yl)propyl][4-(2-methoxy-2-oxoethyl)benzyl]amino}pyrrolidine-1-carboxylate

The subtitle compound was prepared by the method of example 20 step (iii) using the product from step (i).

¹H NMR δ (DMSO-d₆) 7.38-7.14 (9H, m), 6.77 (2H, brs), 5.04 (2H, s), 4.13 (2H, t), 4.02-3.99 (3H, m), 3.84-3.79 (2H, m), 3.67-3.38 (4H, m), 3.65 (2H, s), 3.60 (3H, s), 3.30-3.00 (3H, m), 2.50-2.42 (2H, m), 1.98-1.69 (4H, m), 1.65-1.58 (2H, m), 1.41-1.32 (2H, m), 0.88 (2H, s).

### (iii) Methyl [4-({[3-(6-amino-2-butoxy-8-oxo-7,8-dihydro-9H-purin-9-yl)propyl][(3R)-1-methylpyrrolidin-3-yl]amino}methyl)phenyl]acetate

To a suspension of the product from step (ii) in THF (20 ml) were added 10% Pd/C (513 mg) and NaHCO₃ (500 mg), the mixture was stirred under a H₂ atmosphere at rt for 5.5 h. The reaction mixture was filtered through a Celite pad, which was washed with THF. The filtrate was concentrated and the residue was purified by flash silica gel column chromatography to give the amine (102 mg). To a solution of the amine in MeOH (10 ml) were added formaldehyde aq. (1 ml), NaBH₃CN (130 mg) and AcOH (0.05 ml), and the reaction mixture was stirred at rt for 1 h. The reaction was quenched by satd. NaHCO3 aq., and the mixture was extracted with CHCl₃-MeOH (ca. 20:1). The combined extracts were dried over MgSO4 and concentrated. The residue was purified by flash silica gel column chromatography to afford the amine (82.3 mg) as a colorless oil. To a solution of the amine (80.1 mg) in MeOH (4 ml) was added a solution of 4N HCl-1,4-dioxane (4 ml). After stirring at rt for 5 h, the reaction was quenched by satd. NaHCO₃ aq. (20 ml). The mixture was extracted with CHCl₃-MeOH (ca. 20:1). The combined extracts were dried over MgSO₄ and concentrated to give the title compound (60 mg) as a white solid.

¹H NMR δ (DMSO-d₆) 9.82 (1H, brs), 7.24-7.14 (4H, m), 6.40 (2H, brs), 4.13 (2H, t), 3.68-3.60 (2H, m), 3.63 (2H, s), 3.61 (3H, s), 3.56 (1H, d), 3.44 (1H, d), 2.50-2.36 (4H, m), 2.29-2.20 (1H, m), 2.16 (3H, s), 1.84-1.72 (3H, m), 1.42-1.33 (2H, m), 0.89 (3H, t).

### EXAMPLE 25

### Methyl (4-{[[3-(6-amino-2-butoxy-8-oxo-7,8-dihydro-9H-purin-9-yl)propyl](1′-methyl-1,4′-bipiperidin-4-yl)amino]methyl}phenyl)acetate

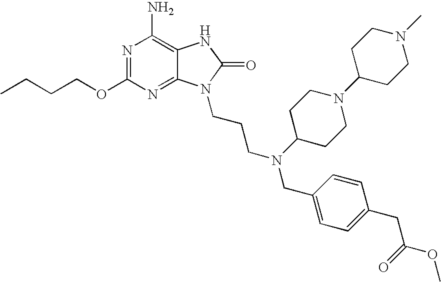

The title compound was prepared using the methods of example 1 using methyl (4-formylphenyl)acetate and example 5 using N-methylpiperidone, yield 41 mg.

¹H NMR δ (DMSO-d₆) 9.81 (1H, brs), 7.24 (2H, d), 7.14 (2H, d), 6.38 (2H, s), 4.12 (2H, t), 3.66-3.60 (7H, m), 3.53 (2H, s), 2.84 (2H, d), 2.75 (2H, d), 2.45 (2H, t), 2.35-2.32 (1H, m), 2.10-2.06 (4H, m), 1.96 (2H, t), 1.79-1.73 (4H, m), 1.65-1.58 (6H, m), 1.41-1.32 (6H, m), 0.90 (3H, t).

### EXAMPLE 26

### Methyl (4-{[[3-(6-amino-2-butoxy-8-oxo-7,8-dihydro-9H-purin-9-yl)propyl](1-propylazetidin-3-yl)amino]methyl}phenyl)acetate

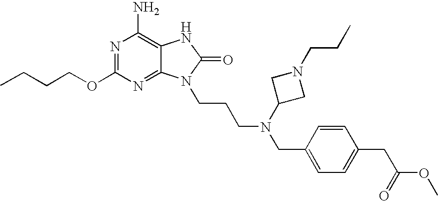

The title compound was prepared by the method of example 20 using isobutylaldehyde.

¹H NMR δ (DMSO-d₆) 9.83 (1H, brs), 7.19-7.12 (4H, m), 6.39 (2H, brs), 4.14 (2H, t), 3.67-3.61 (2H, m), 3.64 (2H, s), 3.61 (3H, s), 3.40 (2H, s), 3.28-3.22 (2H, m), 3.13-3.07 (2H, m), 2.28-2.09 (4H, m), 1.82-1.74 (2H, m), 1.67-1.59 (2H, m), 1.43-1.32 (2H, m), 1.26-1.16 (2H, m), 0.91 (3H, t), 0.80 (3H, t).

### EXAMPLE 27

### Methyl [4-({[3-(6-amino-2-butoxy-8-oxo-7,8-dihydro-9H-purin-9-yl)propyl][(3S)-1-methylpyrrolidin-3-yl]amino}methyl)phenyl]acetate

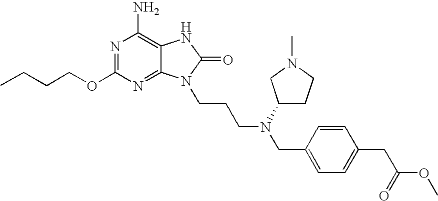

The title compound was prepared by the method of example 24 using (3S)-(3-Boc-amino)pyrrolidine

¹H NMR δ (DMSO-d₆) 9.82 (1H, brs), 7.17-7.11 (4H, m), 6.40 (2H, brs), 4.14 (2H, t), 3.67-3.62 (2H, m), 3.64 (2H, s), 3.60 (3H, s), 3.40 (2H, s), 3.32-3.24 (2H, m), 3.14-3.06 (2H, m), 2.60-2.54 (2H, m), 2.28-2.23 (2H, m), 2.10-2.05 (2H, m), 1.82-1.74 (2H, m), 1.68-1.58 (2H, m), 1.49-1.24 (5H, m), 0.91 (3H, t), 0.94-0.83 (1H, m), 0.78 (6H, d).

### Biological Assay

Human TLR7 Assay

Recombinant human TLR7 was stably expressed in a HEK293 cell line already stably expressing the pNiFty2-SEAP reporter plasmid; integration of the reporter gene was maintained by selection with the antibiotic zeocin. The most common variant sequence of human TLR7 (represented by the EMBL sequence AF240467) was cloned into the mammalian cell expression vector pUNO and transfected into this reporter cell-line. Transfectants with stable expression were selected using the antibiotic blasticidin. In this reporter cell-line, expression of secreted alkaline phosphatase (SEAP) is controlled by an NFkB/ELAM-1 composite promoter comprising five NFkB sites combined with the proximal ELAM-1 promoter. TLR signaling leads to the translocation of NFkB and activation of the promoter results in expression of the SEAP gene. TLR7-specific activation was assessed by determining the level of SEAP produced following overnight incubation of the cells at 37° C. with the standard compound in the presence of 0.1% (v/v) dimethylsulfoxide (DMSO). Concentration dependent induction of SEAP production by compounds was expressed as the log of the minimal effective concentration of compound to induce SEAP release (pMEC).

| | |
|---|---|
| Compound of Example: 10 | pMEC 8.4 |
| Compound of Example: 14 | pMEC 7.7 |
| Compound of Example: 17 | pMEC 9.1 |

1: A compound of formula (I): 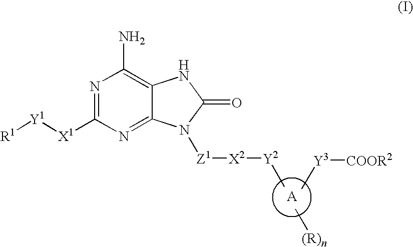
wherein
R¹ represents hydrogen, hydroxyl, C₁-C₆ alkoxy, C₂-C₅ alkoxycarbonyl, C₁-C₆ haloalkyl, C₁-C₆ haloalkoxy, or a C₆-C₁₀ aryl, C₅-C₁₀ heteroaryl or C₃-C₈ cycloalkyl group, each group being optionally substituted by one or more substituents independently selected from halogen, hydroxyl, C₁-C₆ alkyl, C₁-C₆ haloalkyl, C₁-C₆ alkoxy, C₁-C₆ haloalkoxy, C₂-C₅ alkoxycarbonyl, amino (NH₂) and (di)-C₁-C₆ alkylamino;
Y¹ represents a single bond or C₁-C₆ alkylene;
X¹ represents a single bond or an oxygen or sulphur atom or sulphonyl (SO₂) or NR³;
Z¹ represents a C₂-C₆ alkylene or C₃-C₈ cycloalkylene group, each of which may be optionally substituted by at least one hydroxyl;
X² represents NR⁴, CONR⁴, NR⁴CO, SO₂NR⁴, NR⁴SO₂, NR⁴CONR⁵ or NR⁵CON⁴;
Y² represents a single bond or C₁-C₆ alkylene;
Y³ represents a single bond or C₁-C₆ alkylene;
n is an integer 0, 1 or 2;
each R independently represents halogen, C₁-C₆ alkyl, C₁-C₆ hydroxyalkyl, C₁-C₆ haloalkyl, C₁-C₆ alkoxy, C₁-C₆ hydroxyalkoxy, C₁-C₆ haloalkoxy, amino (NH₂), (di)-C₁-C₆ alkylamino, C₁-C₆ alkylamino or a C₄-C₇ saturated heterocyclic ring comprising a ring nitrogen atom and optionally one or more further heteroatoms independently selected from nitrogen, oxygen and sulphur, the heterocyclic ring being optionally substituted by one or more substituents independently selected from halogen, hydroxyl, oxo, C₁-C₆ alkyl, C₁-C₆ alkoxy, C₂-C₅ alkylcarbonyl and C₂-C₅ alkoxycarbonyl;
R² represents hydrogen or a C₁-C₆ alkyl, C₂-C₆ alkenyl, C₂-C₆ alkynyl or C₃-C₈ cycloalkyl group, each group being optionally substituted by one or more substituents independently selected from halogen, hydroxyl, C₁-C₆ alkoxy, C₂-C₁₀ acyloxy, amino (NH₂), (di)-C₁-C₆ alkylamino and a C₄-C₇ saturated heterocyclic ring comprising a ring nitrogen atom and optionally one or more further heteroatoms independently selected from nitrogen, oxygen and sulphur, the heterocyclic ring in turn being optionally substituted by one or more substituents independently selected from halogen, hydroxyl, oxo, C₁-C₆ alkyl, C₁-C₆ alkoxy, C₂-C₅ alkylcarbonyl and C₂-C₅ alkoxycarbonyl;
R³ represents hydrogen or C₁-C₆ alkyl;
R⁴ represents a 3- to 8-membered saturated heterocyclic ring comprising a ring group NR⁶;
R⁵ represents hydrogen or a C₁-C₆ alkyl or C₃-C₆ cycloalkyl group, each of which may be optionally substituted by one or more substituents independently selected from halogen hydroxyl and NR⁷R⁸;
R⁶ represents hydrogen, CO₂R⁹, SO₂R⁹, COR⁹, SO₂NR¹⁰R¹¹, CONR¹⁰R¹¹, a 3- to 8-membered saturated heterocyclic ring comprising a ring group NR⁹, or
(i) a C₆-C₁₀ aryl or C₅-C₁₀ heteroaryl group, each of which may be optionally substituted by one or more substituents independently selected from halogen, cyano, oxo, carboxyl, S(O)ₘR¹², OR¹³, SO₂NR¹³R¹⁴, CONR¹³R¹⁴, NR¹³R¹⁴, NR¹³SO₂R¹², NR¹³CO₂R¹², NR¹³COR¹², C₁-C₆alkyl and C₁-C₃haloalkyl, or
(ii) a C₁-C₆ alkyl C₂-C₆ alkenyl, C₂-C₆ alkynyl or C₃-C₈ cycloalkyl group, each of which may be optionally substituted by one or more substituents independently selected from halogen, cyano, C₃-C₈ cycloalkyl, OR¹⁵, S(O)ₚR¹⁶, CO₂R¹⁷, NR¹⁸R¹⁹, CONR¹⁸R¹⁹, NR¹⁸COR¹⁶, SO₂NR¹⁸R¹⁹, NR¹⁸SO₂R¹⁶ and a group as defined in (i) above;
R⁷ and R⁸ each independently represent hydrogen, C₁-C₆ alkyl or C₃-C₆ cycloalkyl, or
R⁷ and R⁸ together with the nitrogen atom to which they are attached form a 3- to 8-membered saturated heterocyclic ring comprising at least one heteroatom or heterogroup selected from nitrogen, oxygen, sulphur and sulphonyl, the heterocyclic ring being optionally substituted by one or more substituents independently selected from halogen, hydroxyl, carboxyl, cyano, OR²³, S(O)_{q}R²³, NR²⁴R²⁵, C₁-C₆alkyl and C₃-C₈ cycloalkyl;
R¹³, R¹⁴, R¹⁵, R¹⁷, R²⁰, R²¹, R²⁴, R²⁵, R²⁶ and R²⁷ each independently represent hydrogen, C₁-C₆ alkyl or C₃-C₆ cycloalkyl;
R⁹ and R²³ each independently represent a C₁-C₆ alkyl or C₁-C₆ cycloalkyl group, each of which may be optionally substituted by one or more substituents independently selected from halogen, carboxyl, hydroxyl and NR²⁰R²¹;
either R¹⁰ represents hydrogen or a C₁-C₆ alkyl, C₂-C₆ alkenyl, C₂-C₆ alkynyl or C₃-C₈ cycloalkyl group, each of which may be optionally substituted by one or more substituents independently selected from halogens hydroxyl, carboxyl, cyano, OR²³, S(O)_{q}R²³, NR²⁴R²⁵ and C₃-C₈ cycloalkyl, and
R¹¹ represents hydrogen or a C₁-C₆ alkyl or C₃-C₆ cycloalkyl group, each of which may be optionally substituted by one or more substituents independently selected from halogen, hydroxyl and NR²⁶R²⁷, or
R¹⁰ and R¹¹ together with the nitrogen atom to which they are attached form a 3- to 8-membered saturated heterocyclic ring comprising at least one heteroatom or heterogroup selected from nitrogen, oxygen, sulphur and sulphonyl, the heterocyclic ring being optionally substituted by one or more substituents independently selected from halogen, hydroxyl, carboxyl, cyano, OR²³, S(O)_{q}R²³, NR²⁴R²⁵, C₁-C₆ alkyl and C₃-C₈ cycloalkyl;
R¹² represents C₁-C₆ alkyl or C₃-C₆ cycloalkyl;
R¹⁸ and R¹⁹ are defined as for R¹⁰ and R¹¹ respectively;
m, p and q each independently represent an integer 0, 1 or 2; and
A represents a C₆-C₁₀ aryl or a C₅-C₁₂ heteroaryl group;
or a pharmaceutically acceptable salt or solvate thereof.

2. The compound according to claim 1 wherein R¹ represents C₁-C₆ alkoxy.

3. The compound according to claim 1 wherein X¹ and Y¹ both represent a single bond.

4. The compound according to claim 1 wherein Z¹ is C₂-C₆ alkylene.

5. The compound according to claim 1 wherein X² represents NR⁴ where R⁴ is a 4-6-membered saturated heterocyclic ring comprising a ring group NR⁶.

6. The compound according to claim 5 wherein R⁶ is hydrogen, COMe, (CH₂)₂OH, (CH₂)₃OH, methyl, ethyl, CH₂CO₂-t-butyl, CH₂CO₂H, benzyl, CH₂CO₂Me, iso-propyl, iso-butyl, CH₂CN, (CH₂)₂CN, (CH₂)₃CN, (CH₂)₃CO₂butyl or (CH₂)₃CO₂H.

7. The compound according to claim 1 wherein Y² represents C₁-C₆ alkylene.

8. The compound according to claim 1 wherein A represents C₆-C₁₀ aryl.

9. The compound according to claim 1 wherein R is hydrogen.

10. The compound according to claim 1 wherein Y³ represents C₁-C₆ alkylene.

11. The compound according to claim 1 wherein R² represents C₁-C₆ alkyl more preferably methyl.

12. The compound according to claim 1 selected from:
Methyl (3-{[[3-(6-amino-2-butoxy-8-oxo-7,8-dihydro-9H-purin-9-yl)propyl](piperidin-4-yl)amino]methyl}phenyl)acetate,
Methyl [3-({(1-acetylpiperidin-4-yl)[3-(6-amino-2-butoxy-5-oxo-7,8-dihydro-9H-purin-9-yl)propyl]amino}methyl)phenyl]acetate,
Methyl [3-({[3-(6-amino-2-butoxy-8-oxo-7,8-dihydro-9H-purin-9-yl)propyl][1-(2-hydroxyethyl)piperidin-4-yl]amino}methyl)phenyl]acetate,
Methyl [3-({[3-(6-amino-2-butoxy-8-oxo-7,8-dihydro-9H-purin-9-yl)propyl][1-(3-hydroxypropyl)piperidin-4-yl]amino}methyl)phenyl]acetate,
Methyl (3-{[[3-(6-amino-2-butoxy-8-oxo-7,8-dihydro-9H-purin-9-yl)propyl](1-methylpiperidin-4-yl)amino]methyl}phenyl)acetate,
Methyl (3-{[[3-(6-amino-2-butoxy-8-oxo-7,8-dihydro-9H-purin-9-yl)propyl](1-ethylpiperidin-4-yl)amino]methyl}phenyl)acetate,
Methyl [3-({[3-(6-amino-2-butoxy-8-oxo-7,8-dihydro-9H-purin-9-yl)propyl][1-(2-tert-butoxy-2-oxoethyl)piperidin-4-yl]amino}methyl)-phenyl]acetate,
(4-{[3-(6-Amino-2-butoxy-8-oxo-7,8-dihydro-9H-purin-9-yl)propyl][3-(2-methoxy-2-oxoethyl)benzyl]amino}piperidin-1-yl)acetic acid,
Methyl (3-{[[3-(6-amino-2-butoxy-8-oxo-7,8-dihydro-9H-purin-9-yl)propyl](1-benzylpiperidin-4-yl)amino]methyl}phenyl)acetate,
Methyl (3-{[[4-(6-amino-2-butoxy-8-oxo-7,8-dihydro-9H-purin-9-yl)butyl](1-methylpiperidin-4-yl)amino]methyl}phenyl)acetate,
Methyl (3-{2-[[3-(6-amino-2-butoxy-8-oxo-7,8-dihydro-9H-purin-9-yl)propyl](1-methylpiperidin-4-yl)amino]-2-oxoethyl}phenyl)acetate,
Methyl (3-({[[3-(6-amino-2-butoxy-8-oxo-7,8-dihydro-9H-purin-9-yl)propyl][(3R)-1-benzylpyrrolidin-3-yl]amino}methyl)phenyl]acetate,
Methyl (3-{[[3-(6-amino-2-butoxy-8-oxo-7,8-dihydro-9H-purin-9-yl)propyl](1-isopropylpiperidin-4-yl)amino]methyl}phenyl)acetate,
Methyl [3-({[3-(6-amino-2-butoxy-8-oxo-7,8-dihydro-9H-purin-9-yl)propyl][1-(cyanomethyl)piperidin-4-yl]amino}methyl)phenyl]acetate,
Methyl [3′-({[3-(6-amino-2-butoxy-8-oxo-7,8-dihydro-9H-purin-9-yl)propyl][1-(2-cyanoethyl)piperidin-4-yl]amino}methyl)phenyl]acetate,
Methyl [3-({[3-(6-amino-2-butoxy-8-oxo-7,8-dihydro-9H-purin-9-yl)propyl][1-(3-cyanopropyl)piperidin-4-yl]amino}methyl)phenyl]acetate,
tert-Butyl 4-(4-{[3-(6-amino-2-butoxy-8-oxo-7,8-dihydro-9-purin-9-yl)propyl][3-(2-methoxy-2-oxoethyl)benezyl]amino}piperidin-1-yl)butanoate,
4-(4-{[3-(6-Amino-2-butoxy-8-oxo-7,8-dihydro-9H-purin-9-yl)propyl][3-(2-methoxy-2-oxoethyl)benzyl]amino}piperidin-1-yl)butanoic acid,
Methyl (3-({[{3-[6-amino-2-(2-methoxyethoxy)-8-oxo-7,8-dihydro-9H-purin-9-yl]propyl}(1-methylpiperidin-4-yl)amino]methyl}phenyl)acetate,
Methyl (4-{[[3-(6-amino-2-butoxy-8-oxo-7,8-dihydro-9H-purin-9-yl)propyl](1-methylazetidin-3-yl)amino]methyl}phenyl)acetate,
Methyl (4-{[[3-(6-amino-2-butoxy-8-oxo-7,8-dihydro-9H-purin-9-yl)propyl](1-ethylazetidin-3-yl)amino]methyl}phenyl)acetate,
Methyl (4-{[[3-(6-amino-2-butoxy-8-oxo-7,8-dihydro-9H-purin-9-yl)propyl](1-isopropylazetidin-3-yl)amino]methyl}phenyl)acetate,
Methyl (4-{[[3-(6-amino-2-butoxy-8-oxo-7,8-dihydro-9H-purin-9-yl)propyl](1-isobutylazetidin-3-yl)amino]methyl}phenyl)acetate,
Methyl [4-{[[3-(6-amino-2-butoxy-8-oxo-7,8-dihydro-9H-purin-9-yl)propyl][(3R)-1-methylpyrrolidin-3-yl]amino}methyl)phenyl]acetate,
Methyl (4-{[[3-(6-amino-2-butoxy-5-oxo-7,8-dihydro-9H-purin-9-yl)propyl](1′-methyl-1,4′-bipiperidin-4-yl)amino]methyl}phenyl)acetate,
Methyl (4-{[[3-(6-amino-2-butoxy-5-oxo-7,8-dihydro-9H-purin-9-yl)propyl](1-propylazetidin-3-yl)amino]methyl}phenyl)acetate, and
Methyl [4-({[3-(6-amino-2-butoxy-8-oxo-7,8-dihydro-9H-purin-9-yl)propyl][(3S)-1-methylpyrrolidin-3-yl]amino}methyl)phenyl]acetate
and pharmaceutically acceptable salts or solvates thereof.

13. A process for the preparation of a compound of formula (I) where X² represents NR⁴ comprising reacting a compound of formula (II) 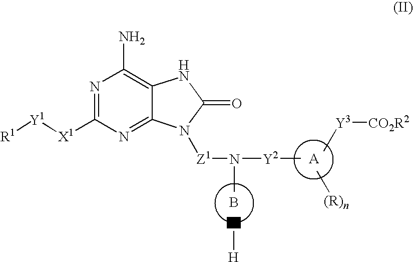
wherein n, Y¹, Y², Y³, X¹, A, Z¹, R, R¹ and R² are as defined in formula (I) and B is defined as a 3- to 8-membered saturated heterocyclic ring comprising a ring group NH, with a compound of formula
L¹-R⁶ (III)
wherein L¹ represents a leaving group (e.g. halogen, mesylate or triflate) and R⁶ is as defined in formula (I),
and optionally after carrying out one or more of the following:
converting the compound obtained to a further compound of the invention
removal of any protecting groups
forming a pharmaceutically acceptable salt of the compound.

14. A pharmaceutical composition comprising a compound of formula (I) or a pharmaceutically acceptable salt thereof as claimed in claim 1 in association with a pharmaceutically acceptable adjuvant, diluent or carrier.

15. A process for the preparation of a pharmaceutical composition as claimed in claim 14 which comprises mixing a compound of formula (I) or a pharmaceutically acceptable salt thereof as claimed in claim of claim 1 with a pharmaceutically acceptable adjuvant, diluent or carrier.

16-19. (canceled)

20. A method of treating, or reducing the risk of a disease or condition in which modulation of TLR7 activity is beneficial which comprises administering to a patient in need thereof a therapeutically effective amount of a compound of formula (I) or a pharmaceutically acceptable salt or solvate thereof as claimed in claim 1.

21. A method of treating, or reducing the risk of, an allergic or viral disease or cancer which comprises administering to a patient in need thereof a therapeutically effective amount of a compound of formula (I) or a pharmaceutically acceptable salt or solvate thereof as claimed in claim 1.

22. A method of treating, or reducing the risk of, an obstructive airways disease or condition which comprises administering to a patient in need thereof a therapeutically effective amount of a compound of formula (I) or a pharmaceutically acceptable salt or solvate thereof as claimed in claim 1.
